(19) United States
(12) Patent Application Publication
(10) Pub. No.: US 2006/0004314 A1
(43) Pub. Date: Jan. 5, 2006

(54) Antimicrobial barriers, systems, and methods formed from hydrophilic polymer structures such as chistosan

(75) Inventors: Simon J. McCarthy, Portland, OR (US); Kenton W. Gregory, Portland, OR (US); John W. Morgan, Portland, OR (US)

Correspondence Address: RYAN KROMHOLZ & MANION, S.C., POST OFFICE BOX 26618, MILWAUKEE, WI, 53226, US

(73) Assignee: HemCon, Inc.

(21) Appl. No.: 11/202,558

(22) Filed: Aug. 12, 2005

Related U.S. Application Data
(63) Continuation-in-part of application No. 11/020,365, filed on Dec. 23, 2004.
Application No. 11/020,365 is a continuation-in-part of application No. 10/743,052, filed on Dec. 23, 2003.
Application No. 10/743,052 is a continuation-in-part of application No. 10/480,827, filed on Oct. 6, 2004.
(60) Provisional application No. 60/298,773, filed on Jun. 14, 2001.

(51) Int. Cl. A61K 31/722; A61F 15/00

(52) U.S. Cl. 602/43; 514/55

(57) ABSTRACT

An antimicrobial barrier comprising a structure including a chitosan biomaterial. The antimicrobial barrier can be used, e.g., (i) stanch, seal, or stabilize a site of tissue injury, tissue trauma, or tissue access; or (ii) form an anti-microbial barrier; or (iii) form an antiviral patch; or (iv) intervene in a bleeding disorder; or (v) release a therapeutic agent; or (vi) treat a mucosal surface; or (vii) combinations thereof. The structure of the antimicrobial barrier may be densified by compression.

### RELATED APPLICATIONS

This application is a continuation-in-part of U.S. Ser. No. 11/020,365, filed on Dec. 23, 2004, entitled “Tissue Dressing Assemblies, Systems and Methods formed from Hydrophilic Polymer Sponge Structures such as Chitosan”, which is a continuation-in-part of U.S. patent application Ser. No. 10/743,052, filed on Dec. 23, 2003, entitled “Wound Dressing and Method of Controlling Severe Life-Threatening Bleeding,” which is a continuation-in-part of U.S. patent application Ser. No. 10/480,827, filed on Oct. 6, 2004, entitled “Wound Dressing and Method of Controlling Severe Life-Threatening Bleeding,” which was a national stage filing under 37 C.F.R. § 371 of International Application No. PCT/US02/18757, filed on Jun. 14, 2002, which claims the benefit of provisional patent application Ser. No. 60/298,773, filed Jun. 14, 2001, which are each incorporated herein by reference.

### BACKGROUND OF THE INVENTION

The application of continuous pressure with gauze bandage remains a primary intervention technique used to stem blood flow, especially flow from severely bleeding wounds. However, this procedure neither effectively nor safely stanches severe blood flow. This has been, and continues to be, a major survival problem in the case of severe life-threatening bleeding from a wound.

Hemostatic bandages such as collagen wound dressings or dry fibrin thrombin wound dressings or chitosan and chitosan dressings are available, such dressings are not sufficiently resistant to dissolution in high blood flow. They also do not possess enough adhesive properties to serve any practical purpose in the stanching of severe blood flow. These currently available surgical hemostatic bandages are also delicate and thus prone to failure should they be damaged by bending or loading with pressure. They are also susceptible to dissolution in hemorrhagic bleeding. Such dissolution and collapse of these bandages may be catastrophic, because it can produce a loss of adhesion to the wound and allow bleeding to continue unabated.

Along with adequately preventing and limiting bleeding and hemorrhaging, care must be taken to prevent bacterial infections from arising on and around the wound or lesion. Current bandages do not adequately prevent the growth of such infections and do not treat such infections.

There remains a need for improved hemostatic dressings with robustness and longevity to resist dissolution during use that will assist in the treatment of bacterial infections.

### SUMMARY OF THE INVENTION

The invention provides antimicrobial barriers, systems and methods formed from a structure including a chitosan biomaterial. The antimicrobial barriers can be used, e.g., (i) to stanch, seal, or stabilize a site of tissue injury, tissue trauma, or tissue access; or (ii) to form an anti-microbial barrier; or (iii) to form an antiviral patch; or (iv) to intervene in a bleeding disorder; or (v) to release a therapeutic agent; or (vi) to treat a mucosal surface; or (vii) combinations thereof.

In one embodiment, the antimicrobial barrier structure is desirably densified by compression.

Other features and advantages of the invention shall be apparent based upon the accompanying description, drawings, and claims.

### DESCRIPTION OF THE DRAWINGS

FIG. 1 is a perspective assembled view of a antimicrobial barrier pad assembly that is capable of adhering to body tissue in the presence of blood, fluid, or moisture.

FIG. 2 is a perspective exploded view of the antimicrobial barrier pad assembly shown in FIG. 1.

FIG. 3 is a perspective view of the antimicrobial barrier pad assembly shown in FIG. 1 packaged in a sealed pouch for terminal irradiation and storage.

FIGS. 4 and 5 are perspective views of the sealed pouch shown in FIG. 3 being torn open to expose the antimicrobial barrier pad assembly for use.

FIGS. 6 and 7 are perspective views of the antimicrobial barrier pad assembly being held and manipulated by folding or bending prior to application to conform to the topology of a targeted tissue site.

FIGS. 8 to 9A/B are perspective views of the antimicrobial barrier pad assembly being applied to a targeted tissue site to stanch bleeding.

FIGS. 10 and 11 are perspective views of pieces of a antimicrobial barrier pad assembly being cut and fitted to a targeted tissue site to stanch bleeding.

FIGS. 12 and 13 are perspective views of the antimicrobial barrier pad assembly being held and manipulated by molding into a concave or cup shape to conform to a targeted tissue site.

FIG. 14 is a diagrammatic view of the steps of a process for creating the antimicrobial barrier pad assembly shown in FIG. 1.

FIGS. 15, 16A/B, and 17A/B are perspective views of an embodiment of the steps for conditioning a hydrophilic polymer structure to create micro-fractures, which provide improved flexibility and compliance.

FIGS. 18A and 18B are views of an embodiment of the steps for conditioning a hydrophilic polymer structure by forming deep relief patterns, which provide improved flexibility and compliance.

FIGS. 19A to 19F are plane views of relief patterns that can be applied to condition a hydrophilic polymer structure following the steps shown in FIGS. 18A and 18B.

FIGS. 20A and 20B are graphs demonstrating the improvement in flexibility and compliance that the treatment steps shown in FIGS. 18A and 18B can provide.

FIGS. 21A and 21B are views of an embodiment of the steps for conditioning a hydrophilic polymer structure by forming vertical channels (perforations), which provide improved flexibility and compliance.

FIG. 22 is a perspective assembled view of a tissue dressing sheet assembly that is capable of adhering to body tissue in the presence of blood, fluid, or moisture.

FIG. 23 is a perspective exploded view of the tissue dressing sheet assembly shown in FIG. 22.

FIG. 24A is a perspective assembled view of tissue dressing sheet assemblies arranged in sheet form.

FIG. 24B is a perspective assembled view of tissue dressing sheet assemblies arranged in roll form.

FIG. 25 is a perspective view of the stuffing of a tissue dressing sheet assembly in roll form into a targeted tissue region to stanch bleeding.

FIGS. 26A to 26F are diagrammatic views of the steps of a process for creating the tissue dressing sheet assembly shown in FIG. 22.

FIG. 27 is a perspective view of the antimicrobial barrier pad assembly shown in FIG. 16 packaged in a sealed pouch for terminal irradiation and storage.

FIG. 28 is a graph demonstrating the flexibility and compliance of a tissue dressing sheet assembly, as shown in FIG. 22, compared to an untreated antimicrobial barrier pad assembly shown in FIG. 1.

FIG. 29A is a graph showing the simulated wound sealing characteristics of a tissue dressing sheet assembly, as shown in FIG. 21 prior to gamma-irradiation.

FIG. 29B is a graph showing the simulated wound sealing characteristics of a tissue dressing sheet assembly, as shown in FIG. 21 before and after gamma-irradiation.

FIG. 30 is a perspective view of a composite tissue dressing assembly that has been shaped and configured to form a gasket assembly to adhere about and seal an access site for an indwelling catheter.

FIG. 31 is a side section view of the gasket assembly shown in FIG. 30.

FIG. 32 is a perspective view of a antimicrobial barrier pad assembly of the type shown in FIG. 1 that has been shaped and configured to form a gasket assembly to adhere about and seal an access site for an indwelling catheter.

FIG. 33 is a perspective view of a tissue dressing sheet assembly of the type shown in FIG. 22 that has been shaped and configured to form a gasket assembly to adhere about and seal an access site for an indwelling catheter.

FIGS. 34 and 35 are graphs showing luminescence detection of a dressing assembly according to the present invention and compared to other available anti-microbial products.

FIGS. 36, 37, and 38 are graphs showing bacterial survival rates of a dressing assembly according to the present invention and compared to other anti-microbial products.

### DESCRIPTION OF THE PREFERRED EMBODIMENT

To facilitate an understanding of this disclosure, the following listing summarizes the topical areas covered, arranged in the order in which they appear:

### List of Topical Areas Described

I. The Antimicrobial Barrier Pad Assembly

A. Overview
1. The Tissue Dressing Matrix
2. The Backing
3. The Pouch

B. Use of the Antimicrobial Barrier Pad Assembly

### EXAMPLE 1

C. Manufacture of the Tissue Dressing Pad Assembly
1. Preparation of a Chitosan Solution
2. Degassing the Aqueous Chitosan Solution
3. Freezing the Aqueous Chitosan Solution
4. Freeze Drying the Chitosan/Ice Matrix
5. Densification of the Chitosan Matrix
6. Securing the Backing
7. Placement in the Pouch
8. Terminal Sterilization

D. Altering the Compliance Properties of a Hydrophilic Polymer Structure
1. Controlled Micro-Fracturing
2. Controlled Macro-Texturing

### EXAMPLE 2

3. Controlled Formation of Vertical Channels
   II. Tissue Dressing Sheet Assembly

A. Overview

B. Use of Tissue Dressing Sheet Assembly

C. Manufacture of the Tissue Dressing Sheet Assembly

### EXAMPLES 3 AND 4

III. Further Indications and Configurations for Hydrophilic Polymer Structures

A. Anti-Microbial Barriers

### EXAMPLES 5 AND 6

IV. Conclusion

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

I. Tissue Dressing Pad Assembly

A. Overview

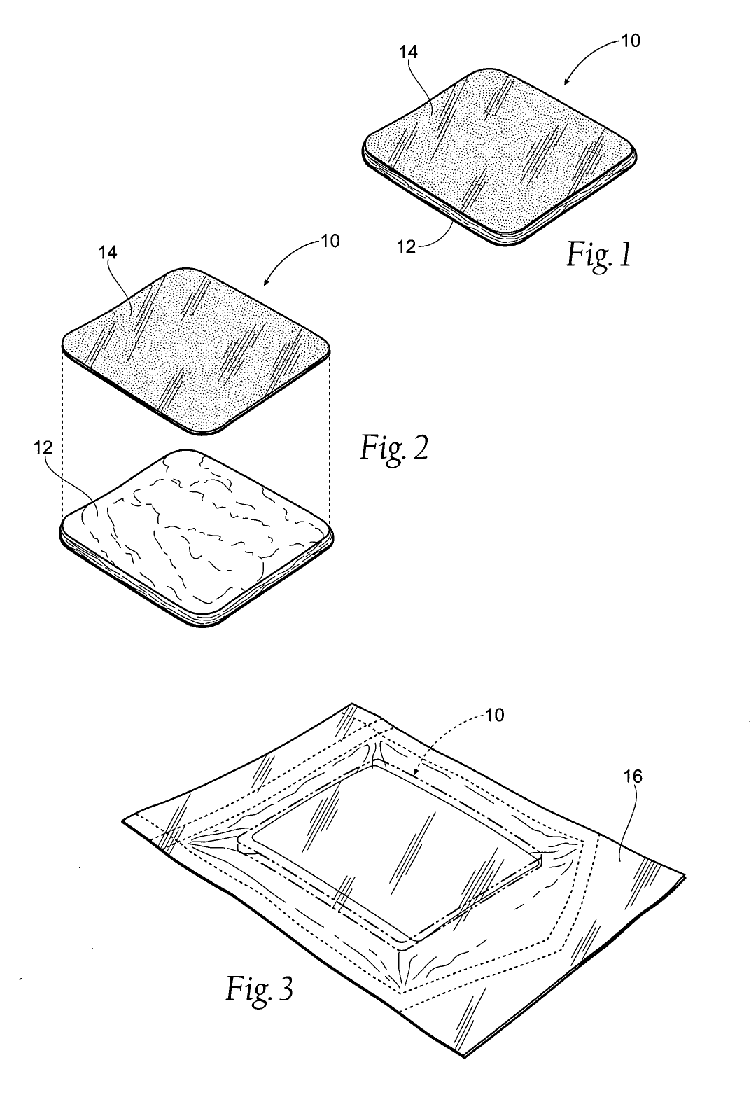

FIG. 1 shows an antimicrobial barrier pad assembly 10. In use, the antimicrobial barrier pad assembly 10 is capable of adhering to tissue in the presence of blood, or body fluids, or moisture. The antimicrobial barrier pad assembly 10 can be used to stanch, seal, and/or stabilize a site of tissue injury, or tissue trauma, or tissue access (e.g., a catheter or feeding tube) against bleeding, fluid seepage or weeping, or other forms of fluid loss. The tissue site treated can comprise, e.g., arterial and/or venous bleeding, or a laceration, or an entrance/entry wound, or a tissue puncture, or a catheter access site, or a burn, or a suture. The antimicrobial barrier pad assembly 10 can also desirably form an anti-bacterial and/or anti-microbial and/or anti-viral protective barrier at or surrounding the tissue treatment site.

FIG. 1 shows the antimicrobial barrier pad assembly 10 in its condition prior to use. As FIG. 2 best shows, the antimicrobial barrier pad assembly 10 comprises a tissue dressing matrix 12 and a pad backing 14 that overlays one surface of the tissue dressing matrix 12. Desirably, the tissue dressing matrix 12 and the backing 14 possess different colors, textures, or are otherwise visually and/or tactilely differentiated, to facilitate recognition by a caregiver.

The size, shape, and configuration of the antimicrobial barrier pad assembly 10 can vary according to its intended use. The pad assembly 10 can be rectilinear, elongated, square, round, oval, or a composite or complex combination thereof. Desirably, as will be described later, the shape, size, and configuration of pad assembly 10 can be formed by cutting, bending, or molding, either during use or in advance of use. In FIG. 1, a representative configuration of the antimicrobial barrier pad assembly 10 is shown that is very useful for the temporary control of external bleeding or fluid loss. By way of example, its size is 10 cm×10 cm×0.55 cm.

1. The Tissue Dressing Matrix

The tissue dressing matrix 12 is preferably formed from a low modulus hydrophilic polymer matrix, i.e., an inherently “uncompressed” tissue dressing matrix 12, which has been densified by a subsequent densification process, which will be described later. The tissue dressing matrix 12, preferably, includes a biocompatible material that reacts in the presence of blood, body fluid, or moisture to become a strong adhesive or glue. Desirably, the tissue dressing matrix also possesses other beneficial attributes, for example, anti-bacterial and/or anti-microbial anti-viral characteristics, and/or characteristics that accelerate or otherwise enhance the body's defensive reaction to injury.

The tissue dressing matrix 12 may comprise a hydrophilic polymer form, such as a polyacrylate, an alginate, chitosan, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof. The starch may be of amylase, amylopectin and a combination of amylopectin and amylase.

In a preferred embodiment, the biocompatible material of the matrix 12 comprises a non-mammalian material, which is most preferably poly [β-(1→4)-2-amino-2-deoxy-D-glucopyranose, which is more commonly referred to as chitosan. The chitosan selected for the matrix 12 preferably has a weight average molecular weight of at least about 100 kDa, and more preferably, of at least about 150 kDa. Most preferably, the chitosan has a weight average molecular weight of at least about 300 kDa.

In forming the matrix 12, the chitosan is desirably placed into solution with an acid, such as glutamic acid, lactic acid, formic acid, hydrochloric acid and/or acetic acid. Among these, hydrochloric acid and acetic acid are most preferred, because chitosan acetate salt and chitosan chloride salt resist dissolution in blood whereas chitosan lactate salt and chitosan glutamate salt do not. Larger molecular weight (Mw) anions disrupt the para-crystalline structure of the chitosan salt, causing a plasticization effect in the structure (enhanced flexibility). Undesirably, they also provide for rapid dissolution of these larger Mw anion salts in blood.

One preferred form of the matrix 12 comprises an “uncompressed” chitosan acetate matrix 12 of density less than 0.035 g/cm³ that has been formed by freezing and lyophilizing a chitosan acetate solution, which is then densified by compression to a density of from 0.6 to 0.25 g/cm³, with a most preferred density of about 0.20 g/cm³. This chitosan matrix 12 can also be characterized as a compressed, hydrophilic structure. The densified chitosan matrix 12 exhibits all of the above-described characteristics deemed to be desirable. It also possesses certain structural and mechanical benefits that lend robustness and longevity to the matrix during use, as will be described in greater detail later.

The chitosan matrix 12 presents a robust, permeable, high specific surface area, positively charged surface. The positively charged surface creates a highly reactive surface for red blood cell and platelet interaction. Red blood cell membranes are negatively charged, and they are attracted to the chitosan matrix 12. The cellular membranes fuse to chitosan matrix 12 upon contact. A clot can be formed very quickly, circumventing immediate need for clotting proteins that are normally required for hemostasis. For this reason, the chitosan matrix 12 is effective for both normal as well as anti-coagulated individuals, and as well as persons having a coagulation disorder like hemophilia. The chitosan matrix 12 also binds bacteria, endotoxins, and microbes, and can kill bacteria, microbes, and/or viral agents on contact.

Further details of the structure, composition, manufacture, and other technical features of the chitosan matrix 12 will be described later.

2. The Backing

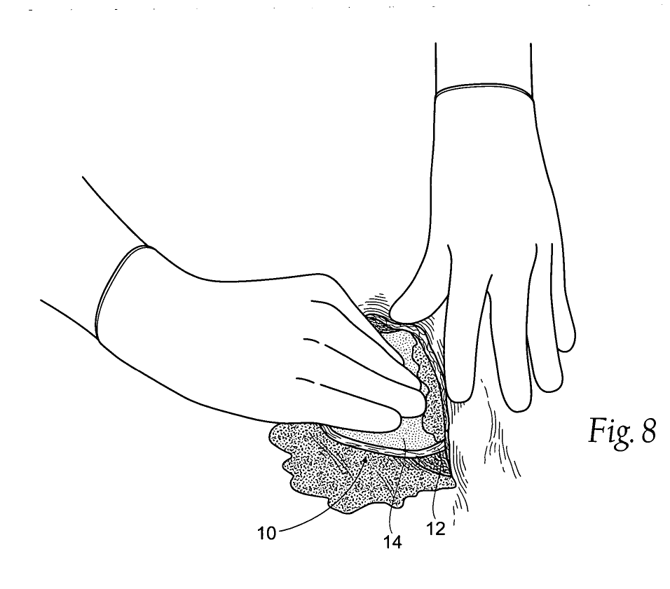

The tissue dressing pad assemble is sized and configured for manipulation by a caregiver's fingers and hand. The backing 14 isolates a caregiver's fingers and hand from the fluid-reactive chitosan matrix 12 (see, e.g., FIG. 8). The backing 14 permits the chitosan matrix 12 to be handled, manipulated, and applied at the tissue site, without adhering or sticking to the caregiver's fingers or hand. The backing 14 can comprise low-modular meshes and/or films and/or weaves of synthetic and naturally occurring polymers. In a preferred embodiment for temporary external wound applications, the backing 14 comprises a fluid impermeable polymeric material, e.g., polyethylene (3M 1774T polyethylene foam medical tape, 0.056 cm thick), although other comparable materials can be used.

Other polymers suitable for backing use in temporary wound applications include, but are not limited to, cellulose polymers, polyethylene, polypropylene, metallocene polymers, polyurethanes, polyvinylchloride polymers, polyesters, polyamides or combinations thereof.

For internal wound applications, a resorbable backing may be used in hydrophilic sponge bandage forms. Preferably such bandage forms would use a biodegradable, biocompatible backing material. Synthetic biodegradable materials may include, but are not limited to, poly(glycolic acid), poly(lactic acid), poly(e-caprolactone), poly(β-hydroxybutyric acid), poly (β-hydroxyvaleric acid), polydioxanone, poly(ethylene oxide), poly(malic acid), poly(tartronic acid), polyphosphazene, copolymers of polyethylene, copolymers of polypropylene, and the copolymers of the monomers used to synthesize the above-mentioned polymers or combinations thereof. Naturally occurring biodegradable polymers may include, but are not limited to, chitin, algin, starch, dextran, collagen and albumen.

3. The Pouch

As FIG. 3 shows, the chitosan matrix 12 is desirably vacuum packaged before use with low moisture content, preferably 5% moisture or less, in an air-tight heat sealed foil-lined pouch 16. The antimicrobial barrier pad assembly 10 is subsequently terminally sterilized within the pouch 16 by use of gamma irradiation.

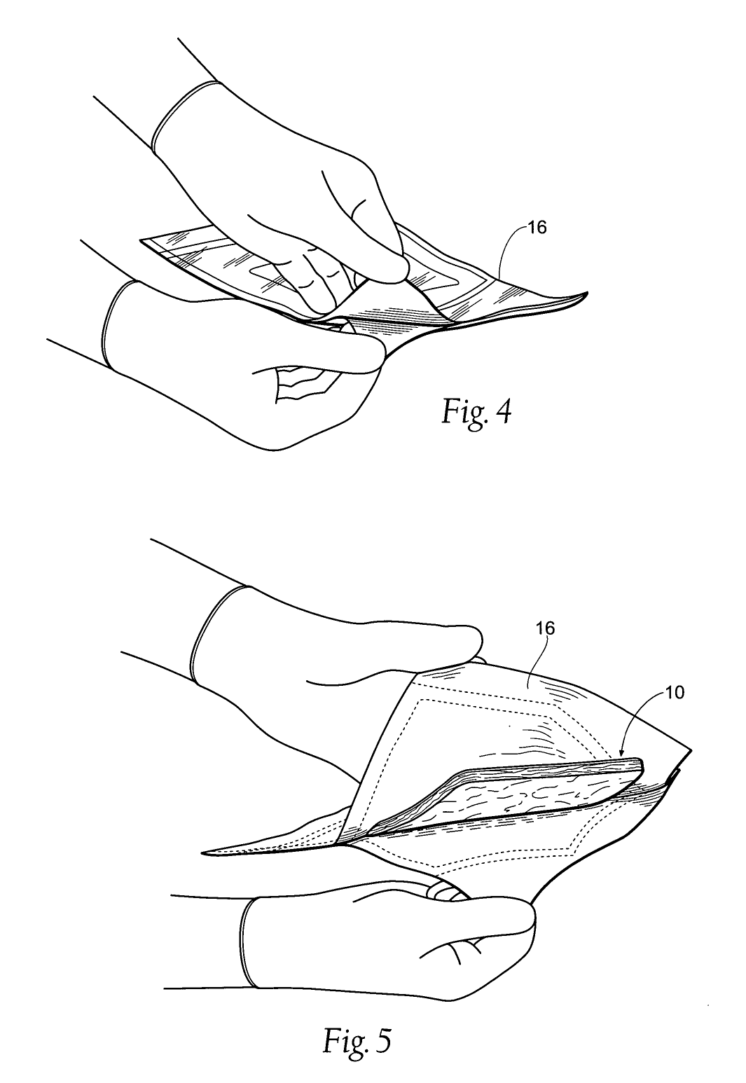

The pouch 16 is configured to be peeled opened by the caregiver (see FIGS. 4 and 5) at the instant of use. The pouch 16 provides peel away access to the antimicrobial barrier pad assembly 10 along one end. The opposing edges of the pouch 16 are grasped and pulled apart to expose the antimicrobial barrier pad assembly 10 for use.

B. Use of the Antimicrobial Barrier Pad Assembly 10

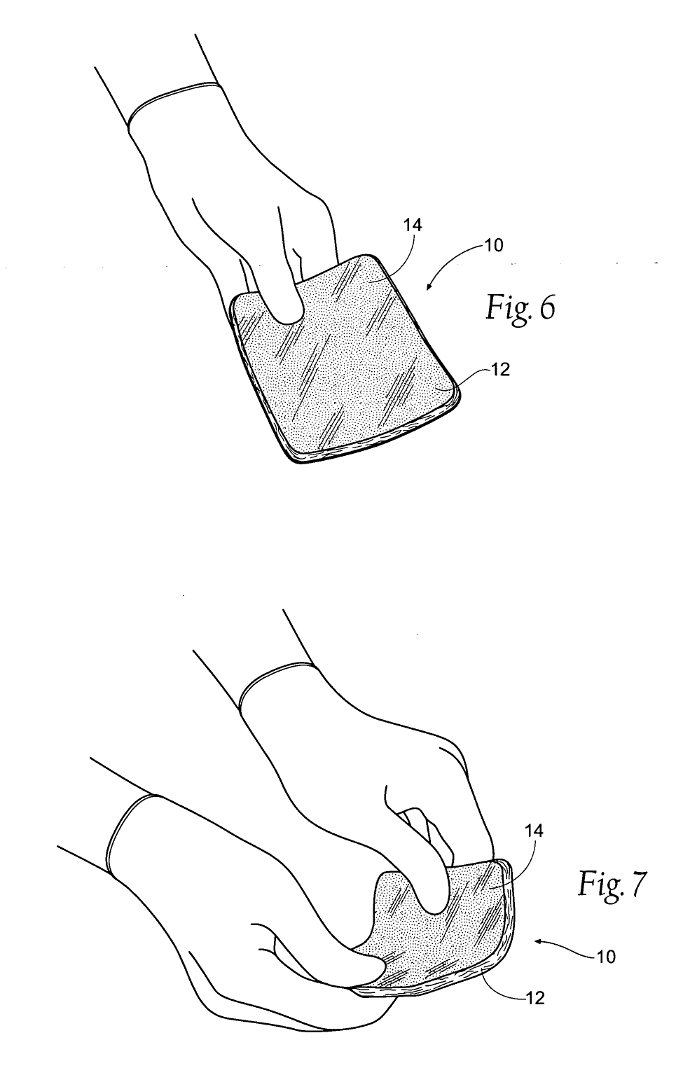

Once removed from the pouch 16 (see FIG. 6), the antimicrobial barrier pad assembly 10 is immediately ready to be adhered to the targeted tissue site. It needs no pre-application manipulation to promote adherence. For example, there is no need to peel away a protective material to expose an adhesive surface for use. The adhesive surface forms in situ, because the chitosan matrix 12 itself exhibits strong adhesive properties once in contact with blood, fluid, or moisture.

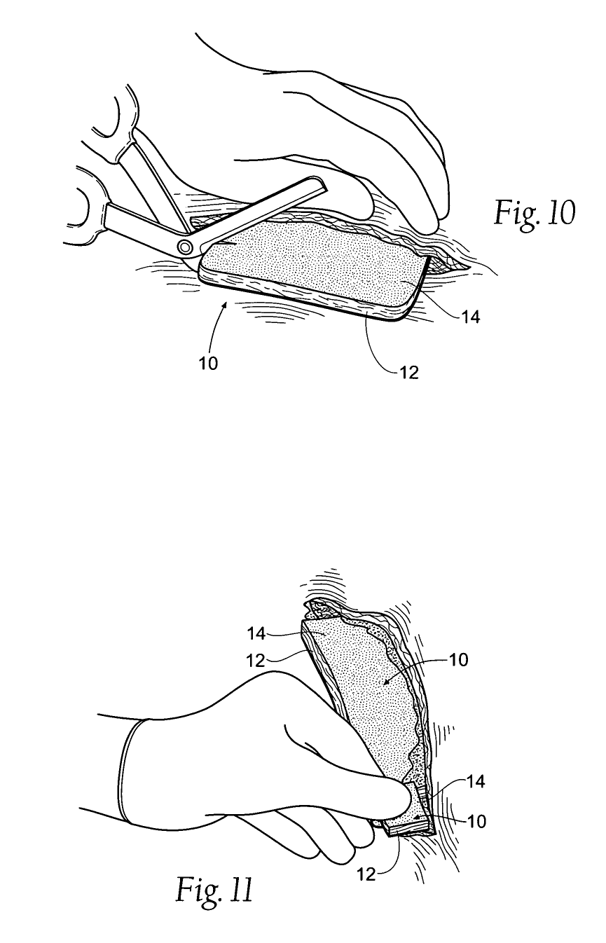

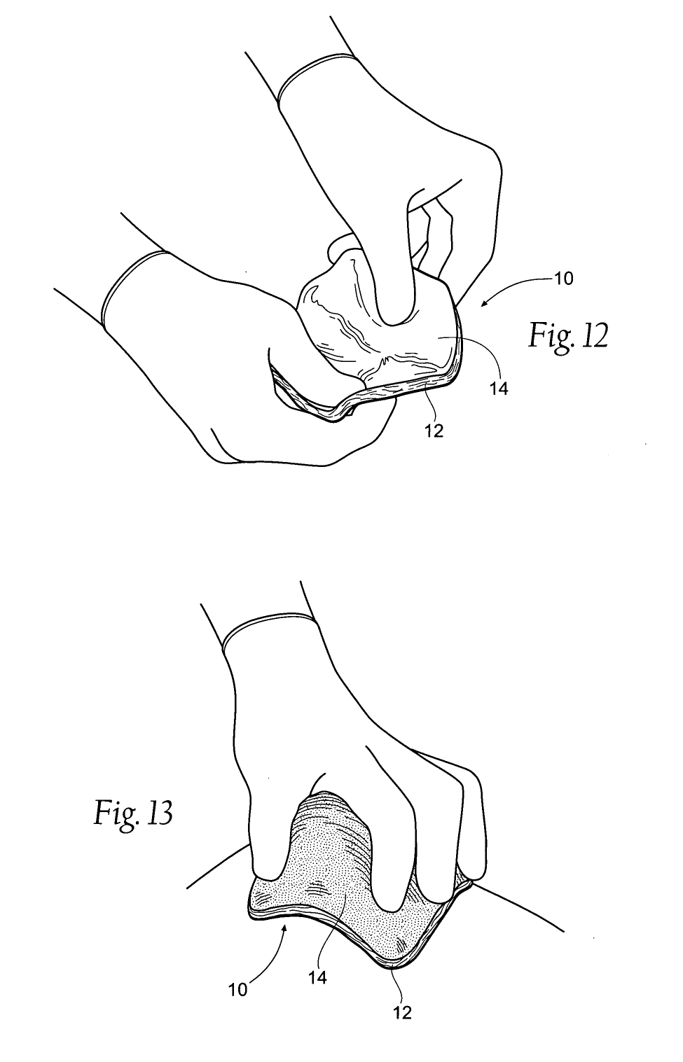

Desirably, the antimicrobial barrier pad assembly 10 is applied to the injury site within one hour of opening the pouch 16. As FIG. 7 shows, the antimicrobial barrier pad assembly 10 can be pre-shaped and adapted on site to conform to the topology and morphology of the site. As FIGS. 11 and 12 show, the antimicrobial barrier pad assembly 10 can be deliberately molded into other configurations, e.g., into a cup-shape, to best conform to the particular topology and morphology of the treatment site. While shaping or otherwise manipulating the antimicrobial barrier pad assembly 10 prior to placement on a treatment site, the caregiver should avoid contact between hand or finger moisture and the chitosan matrix 12. This could cause the chitosan matrix 12 to become sticky and difficult to handle. This is the primary purpose of the backing 14, although the backing 14 also lends added mechanical support and strength to the matrix.

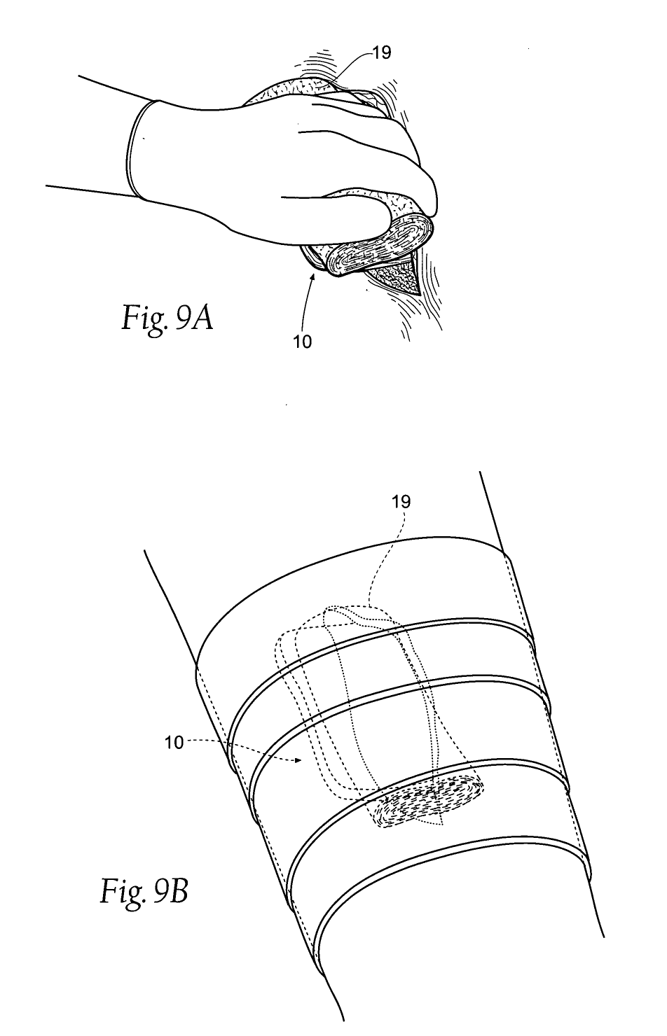

Desirably, as FIG. 8 shows, firm pressure is applied for at least two minutes, to allow the natural adhesive activity of the chitosan matrix 12 to develop. The adhesive strength of the chitosan matrix 12 will increase with duration of applied pressure, up to about five minutes. Even pressure applied across the antimicrobial barrier pad assembly 10 during this time will provide more uniform adhesion and wound sealing. Applying pressure with a Kerlix roll 18 (see FIG. 9A) has been shown to be very effective.

Due to unique mechanical and adhesive characteristics, two or more dressing pad assemblies can be overlapped, if needed, to occupy the wound or tissue site. The chitosan matrix 12 of one pad assembly 10 will adhere to the backing 14 of an adjacent dressing pad assembly 10.

The dressing pad assembly 10 can also be torn or cut on site (see FIG. 10) to match the size of the wound or tissue site. It is desirable to allow at least a one-half inch larger perimeter of the dressing pad assembly 10 over the wound or tissue site to provide good tissue adhesion and sealing. Smaller, patch pieces of a dressing assembly can also be cut to size on site (see FIG. 11), fitted and adhered to the periphery of another pad assembly 10 to best approximate the topology and morphology of the treatment site.

If the tissue pad dressing assembly fails to stick to the injury site, it can be removed and discarded, and another fresh dressing pad assembly 10 applied. In wounds with substantial tissue disruptions, with deep tissue planes or in penetrating wounds, peeling away the backing 14 and stuffing the chitosan matrix 12 into the wound, followed by covering the wound with a second dressing, has been shown to be very effective.

Once pressure has been applied for two to five minutes, and/or control of the bleeding has been accomplished with good dressing adhesion and coverage of the wound or tissue site, a second conventional dressing (e.g., gauze) is desirably applied to secure the dressing and to provide a clean barrier for the wound (see FIG. 9B). If the wound is to be subsequently submersed underwater, a water tight covering should be applied to prevent the dressing from becoming over-hydrated.

Desirably, in the case of FDA cleared temporary dressing forms, the antimicrobial barrier pad assembly 10 is removed within forty-eight hours of application for definitive surgical repair. The antimicrobial barrier pad assembly 10 can be peeled away from the wound and will generally separate from the wound in a single, intact dressing. In some cases, residual chitosan gel may remain, and this can be removed using saline or water with gentle abrasion and a gauze dressing. Chitosan is biodegradable within the body and is broken down into glucosamine, a benign substance. Still, it is desirable in the case of temporary dressings, that efforts should be made to remove all portions of chitosan from the wound at the time of definitive repair. As before discussed, biodegradable dressings can be formed for internal use.

### EXAMPLE 1

### Usage Action Reports

Action reports by combat medics in operations in and during freedom operations in Afghanistan and Iraq have shown successful clinical utility for the dressing pad assemblies without adverse effects. The US Army Institute for Surgical Research at Fort Sam Houston in Texas evaluated the dressing pad assembly 10 in trauma models with severe life threatening bleeding and compared this dressing to standard 4×4 inch cotton gauze dressings. The antimicrobial barrier pad assembly 10 significantly decreased blood loss and decreased resuscitative fluid requirements. Survival at one hour was increased in the group to which the antimicrobial barrier pad assembly 10 was applied, compared to the cotton gauze survival group. Combat medics have successfully treated bullet wounds, shrapnel, land mine and other injuries, when conventional wound dressings have failed.

C. Manufacture of the Tissue Dressing Pad Assembly

A desirable methodology for making the antimicrobial barrier pad assembly 10 will now be described. This methodology is shown schematically in FIG. 16. It should be realized, of course, that other methodologies can be used.

1. Preparation of a Chitosan Solution

The chitosan used to prepare the chitosan solution preferably has a fractional degree of deacetylation greater than 0.78 but less than 0.97. Most preferably the chitosan has a fractional degree of deacetylation greater than 0.85 but less than 0.95. Preferably the chitosan selected for processing into the matrix has a viscosity at 25° C. in a 1% (w/w) solution of 1% (w/w) acetic acid (AA) with spindle LVI at 30 rpm, which is about 100 centipoise to about 2000 centipoise. More preferably, the chitosan has viscosity at 25° C. in a 1% (w/w) solution of 1% (w/w) acetic acid (AA) with spindle LVI at 30 rpm, which is about 125 centipoise to about 1000 centipoise. Most preferably, the chitosan has viscosity at 25° C. in a 1% (w/w) solution of 1% (w/w) acetic acid (AA) with spindle LV1 at 30 rpm, which is about 400 centipoise to about 800 centipoise.

The chitosan solution is preferably prepared at 25° C. by addition of water to solid chitosan flake or powder and the solid dispersed in the liquid by agitation, stirring or shaking. On dispersion of the chitosan in the liquid, the acid component is added and mixed through the dispersion to cause dissolution of the chitosan solid. The rate of dissolution will depend on the temperature of the solution, the molecular weight of the chitosan and the level of agitation. Preferably the dissolution step is performed within a closed tank reactor with agitating blades or a closed rotating vessel. This ensures homogeneous dissolution of the chitosan and no opportunity for high viscosity residue to be trapped on the side of the vessel. Preferably the chitosan solution percentage (w/w) is greater than 0.5% chitosan and less than 2.7% chitosan. More preferably the chitosan solution percentage (w/w) is greater than 1% chitosan and less than 2.3% chitosan. Most preferably the chitosan solution percentage is greater than 1.5% chitosan and less than 2.1% chitosan. Preferably the acid used is acetic acid. Preferably the acetic acid is added to the solution to provide for an acetic acid solution percentage (w/w) at more than 0.8% and less than 4%. More preferably the acetic acid is added to the solution to provide for an acetic acid solution percentage (w/w) at more than 1.5% (w/w) and less than 2.5%.

The structure or form producing steps for the chitosan matrix 12 are typically carried out from solution and can he accomplished employing techniques such as freezing (to cause phase separation), non-solvent die extrusion (to produce a filament), electro-spinning (to produce a filament), phase inversion and precipitation with a non-solvent (as is typically used to produce dialysis and filter membranes) or solution coating onto a preformed sponge-like or woven product. In the case of freezing, where two or more distinct phases are formed by freezing (typically water freezing into ice with differentiation of the chitosan biomaterial into a separate solid phase), another step is required to remove the frozen solvent (typically ice), and hence produce the chitosan matrix 12 without disturbing the frozen structure. This may be accomplished by a freeze-drying and/or a freeze substitution step. The filament can he formed into a non-woven sponge-like mesh by non-woven spinning processes. Alternately, the filament may he produced into a felted weave by conventional spinning and weaving processes. Other processes that may be used to make the biomaterial sponge-like product include dissolution of added porogens from a solid chitosan matrix 12 or boring of material from said matrix.

2. Degassing the Aqueous Chitosan Solution

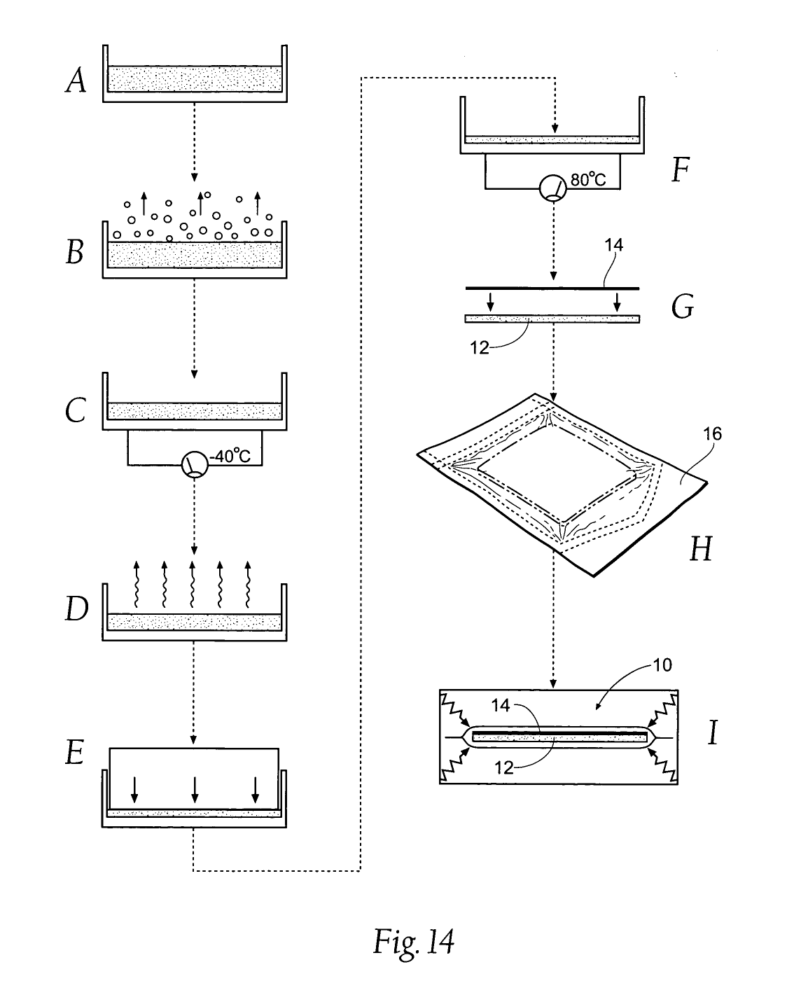

Preferably (see FIG. 14, Step B), the chitosan biomaterial is degassed of general atmospheric gases. Typically, degassing is removing sufficient residual gas from the chitosan biomaterial so that, on undergoing a subsequent freezing operation, the gas does not escape and form unwanted large voids or large trapped gas bubbles in the subject wound dressing product. The degassing step may be performed by heating a chitosan biomaterial, typically in the form of a solution, and then applying a vacuum thereto. For example, degassing can be performed by heating a chitosan solution to about 45° C. immediately prior to applying vacuum at about 500 mTorr for about 5 minutes while agitating the solution.

In one embodiment, certain gases can be added back into the solution to controlled partial pressures after initial degassing. Such gases would include but are not limited to argon, nitrogen and helium. An advantage of this step is that solutions containing partial pressures of these gases form micro-voids on freezing. The microvoid is then carried through the sponge as the ice-front advances. This leaves a well defined and controlled channel that aids sponge pore interconnectivity.

3. Freezing the Aqueous Chitosan Solution

Next (see FIG. 14, Step C), the chitosan biomaterial—which is typically now in acid solution and degassed, as described above—is subjected to a freezing step. Freezing is preferably carried out by cooling the chitosan biomaterial solution supported within a mold and lowering the solution temperature from room temperature to a final temperature below the freezing point. More preferably this freezing step is performed on a plate freezer whereby a thermal gradient is introduced through the chitosan solution in the mold by loss of heat through the plate cooling surface. Preferably this plate cooling surface is in good thermal contact with the mold. Preferably the temperature of the chitosan solution and mold before contact with the plate freezer surface are near room temperature. Preferably the plate freezer surface temperature is not more than −10° C. before introduction of the mold+solution. Preferably the thermal mass of the mold+solution is less than the thermal mass of the plate freezer shelf+heat transfer fluid. Preferably the molds are formed from, but are not limited to, a metallic element such as iron, nickel, silver, copper, aluminum, aluminum alloy, titanium, titanium alloy, vanadium, molybdenum, gold, rhodium, palladium, platinum and/or combinations thereof. The molds may also be coated with thin, inert metallic coatings such as titanium, chromium, tungsten, vanadium, nickel, molybdenum, gold and platinum in order to ensure there is no reaction with the acid component of the chitosan solution and the chitosan salt matrix. Thermally insulating coatings or elements may be used in conjunction with the metallic molds to control heat transfer in the molds. Preferably the mold surfaces do not bind with the frozen chitosan solution. The inside surface of the mold is preferably coated with a thin, permanently-bound, fluorinated release coating formed from polytetrafluoroethylene (Teflon), fluorinated ethylene polymer (FEP), or other fluorinated polymeric materials. Although coated metallic molds are preferable, thin walled plastic molds can be a convenient alternative for supporting the solution. Such plastic molds would include, but not be limited to, molds prepared by injection molding, machining or thermoforming from polyvinylchloride, polystyrene, acrylonitrile-butadiene-styrene copolymers, polyesters, polyamides, polyurethanes and polyolefins. An advantage of the metallic molds combined with local placement of thermally insulating elements is that they also provide opportunity for improved control of heat flow and structure within the freezing sponge. This improvement in heat flow control results from large thermal conductivity differences between thermally conducting and thermally insulating element placements in the mold.

Freezing of the chitosan solution in this way enables the preferred structure of the wound-dressing product to be prepared.

As will be demonstrated below, the plate freezing temperature affects the structure and mechanical properties of the final chitosan matrix 12. The plate freezing temperature is preferably not higher than about −10° C., more preferably not more than about −20° C., and most preferably not more than about −30° C. When frozen at −10° C., the structure of the uncompressed chitosan matrix 12 is very open and vertical throughout the open sponge structure. When frozen at −25° C., the structure of the uncompressed chitosan matrix 12 is more closed, but it is still vertical. When frozen at −40° C., the structure of the uncompressed chitosan matrix 12 is closed and not vertical. Instead, the chitosan matrix 12 comprises more of a reinforced, inter-meshed structure. The adhesive/cohesive sealing properties of the chitosan matrix 12 are observed to improve as lower freezing temperatures are used. A freezing temperatures of about −40° C. forms a structure for the chitosan matrix 12 having superior adhesive/cohesive properties.

During the freezing step, the temperature may be lowered over a predetermined time period. For example, the freezing temperature of a chitosan biomaterial solution may he lowered from room temperature to −45° C. by plate cooling application of a constant temperature cooling ramp of between about −0.4° C./mm to about −0.8° C./mm for a period of about 90 minutes to about 160 minutes.

4. Freeze Drying the Chitosan/Ice Matrix

The frozen chitosan/ice matrix desirably undergoes water removal from within the interstices of the frozen material (see FIG. 14, Step D). This water removal step may he achieved without damaging the structural integrity of the frozen chitosan biomaterial. This may be achieved without producing a liquid phase, which can disrupt the structural arrangement of the ultimate chitosan matrix 12. Thus, the ice in the frozen chitosan biomaterial passes from a solid frozen phase into a gas phase (sublimation) without the formation of an intermediate liquid phase. The sublimated gas is trapped as ice in an evacuated condenser chamber at substantially lower temperature than the frozen chitosan biomaterial.

The preferred manner of implementing the water removal step is by freeze-drying, or lyophilization. Freeze-drying of the frozen chitosan biomaterial can be conducted by further cooling the frozen chitosan biomaterial. Typically, a vacuum is then applied. Next, the evacuated frozen chitosan material may be gradually heated.

More specifically, the frozen chitosan biomaterial may be subjected to subsequent freezing preferably at about −15° C., more preferably at about −25° C., and most preferably at about −45° C., for a preferred time period of at least about 1 hour, more preferably at least about 2 hour, and most preferably at least about 3 hour. This step can be followed by cooling of the condenser to a temperature of less than about −45° C., more preferably at about −60° C., and most preferably at about −85° C. Next, a vacuum in the amount of preferably at most about 100 mTorr, more preferably at most about 150 mTorr, and most preferably at least about 200 mTorr, can be applied. The evacuated frozen chitosan material can be heated preferably at about −25° C., more preferably at about −15° C., and most preferably at about −10° C., for a preferred time period of at least about 1 hour, more preferably at least about 5 hour, and most preferably at least about 10 hour.

Further freeze drying, maintaining vacuum pressure at near 200 mTorr, is conducted at a shelf temperature of about 20° C., more preferably at about 15° C., and most preferably at about 10° C., for a preferred time period of at least about 36 hours, more preferably at least about 42 hours, and most preferably at least about 48 hours.

5. Densification of the Chitosan Matrix

The chitosan matrix before densification (density near 0.03 g/cm³) will be called an “uncompressed chitosan matrix.” This uncompressed matrix is ineffective in stanching bleeding since it rapidly dissolves in blood and has poor mechanical properties. The chitosan biomaterial is necessarily compressed (see FIG. 16, Step E). Compression loading normal to the hydrophilic matrix polymer surface with heated platens can be used to compress the dry “uncompressed” chitosan matrix 12 to reduce the thickness and increase the density of the matrix. The compression step, which will sometimes be called in shorthand “densification,” significantly increases adhesion strength, cohesion strength and dissolution resistance of the chitosan matrix 12. Appropriately frozen chitosan matrices 12 compressed above a threshold density (close to 0.1 g/cm³) do not readily dissolve in flowing blood at 37° C.

The compression temperature is preferably not less than about 60° C., more preferably it is not less than about 75° C. and not more than about 85° C.

After densification, the density of the matrix 12 can be different at the base (“active”) surface of the matrix 12 (i.e., the surface exposed to tissue) than at the top surface of the matrix 12 (the surface to which the backing 14 is applied). For example, in a typical matrix 12 where the mean density measured at the active surface is at or near the most preferred density value of 0.2 g/cm³, the mean density measured at the top surface can be significantly lower, e.g., at 0.05 g/cm³. The desired density ranges as described herein for a densified matrix 12, are intended to exist at are near the active side of the matrix 12, where exposure to blood, fluid, or moisture first occurs.

The densified chitosan biomaterial is next preferably preconditioned by heating chitosan matrix 12 in an oven to a temperature of preferably up to about 75° C., more preferably to a temperature of up to about 80° C., and most preferably to a temperature of preferably up to about 85° C. (FIG. 14, Step F). Preconditioning is typically conducted for a period of time up to about 0.25 hours, preferably up to about 0.35 hours, more preferably up to about 0.45 hours, and most preferably up to about 0.50 hours. This pre-conditioning step provides further significant improvement in dissolution resistance with a small cost in a 20-30% loss of adhesion properties.

6. Secure the Backing to the Densified Chitosan Matrix

The backing 14 is secured to the chitosan matrix 12 to form the antimicrobial barrier pad assembly 10 (see FIG. 14, Step G). The backing 14 can be attached or bonded by direct adhesion with a top layer of chitosan matrix 12. Alternatively, an adhesive such as 3M 9942 Acrylate Skin Adhesive, or fibrin glue, or cyanoacrylate glue can he employed.

7. Placement in the Pouch

The antimicrobial barrier pad assembly 10 can he subsequently packaged in the pouch 16 (see FIG. 14, Step H), which is desirably purged with an inert gas such as either argon or nitrogen gas, evacuated and heat sealed. The pouch 16 acts to maintain interior contents sterility over an extend time (at least 24 months) and also provides a very high barrier to moisture and atmospheric gas infiltration over the same period.

8. Sterilization

After pouching, the processed antimicrobial barrier pad assembly 10 is desirably subjected to a sterilization step (see FIG. 14, Step I). The antimicrobial barrier pad assembly 10 can be sterilized by a number of methods. For example, a preferred method is by irradiation, such as by gamma irradiation, which can further enhance the blood dissolution resistance, the tensile properties and the adhesion properties of the wound dressing. The irradiation can be conducted at a level of at least about 5 kGy, more preferably a least about 10 kGy, and most preferably at least about 15 kGy.

D. Altering the Compliance Properties of a Hydrophilic Polymer Structure

Immediately prior to use, the antimicrobial barrier pad assembly 10 is removed from its pouch 16 (as shown in FIGS. 4 to 6). Due to its low moisture content, the antimicrobial barrier pad assembly 10, upon removed from the pouch 16, can seem to be relatively inflexible and may not immediately mate well with curved and irregular surfaces of the targeted injury site. Bending and/or molding of the pad assembly 10 prior to placement on the targeted injury site has been already described and recommended. The ability to shape the pad assembly 10 is especially important when attempting to control strong bleeding, since apposition of the pad assembly 10 immediately against an injured vessel is necessary to control severe bleeding. Generally, these bleeding vessels are deep within irregularly shaped wounds.

In hydrophilic polymer sponge structure, of which the pad assembly 10 is but one example, the more flexible and compliant the structure is, the more resistant it is to tearing and fragmentation as the structure is made to conform to the shape of the wound and achieve apposition of the sponge structure with the underlying irregular surface of the injury. Resistance to tearing and fragmentation is a benefit, as it maintains wound sealing and hemostatic efficacy. Compliance and flexibility provide an ability to load a hydrophilic polymer sponge structure (e.g., the pad assembly 10) against a deep or crevice shaped wound without cracking or significant pad assembly 10 dissolution.

Improved flexibility and compliance by the use of certain plasticizing agents in solution with the chitosan may be problematic, because certain plasticizers can change other structural attributes of the pad assembly 10. For example, chitosan glutamate and chitosan lactate are more compliant than chitosan acetate. However, glutamate and lactate chitosan acid salts rapidly dissolve in the presence of blood, while the chitosan acetate salt does not. Thus, improved compliance and flexibility can be offset by reduced robustness and longevity of resistance to dissolution.

Improved compliance and flexibility can be achieved by mechanical manipulation of any hydrophilic polymer sponge structure after manufacture, without loss of beneficial features of robustness and longevity of resistance to dissolution. Several ways in which such mechanical manipulation can be accomplished after manufacture will now be described. While the methodologies are described in the context of the chitosan matrix 12, it should be appreciated that the methodologies are broadly applicable for use with any form of hydrophilic polymer sponge structure, of which the chitosan matrix 12 is but one example.

1. Controlled Micro-Fracturing of a Hydrophilic Polymer Sponge Structure

Controlled micro-fracturing of the substructure of a hydrophilic polymer sponge structure such as the chitosan matrix 12 can be accomplished by systematic mechanical pre-conditioning of the dry pad assembly 10. This form of controlled mechanical pre-conditioning of the pad assembly 10 can achieve improved flexibility and compliance, without engendering gross failure of the pad assembly 10 at its time of use.

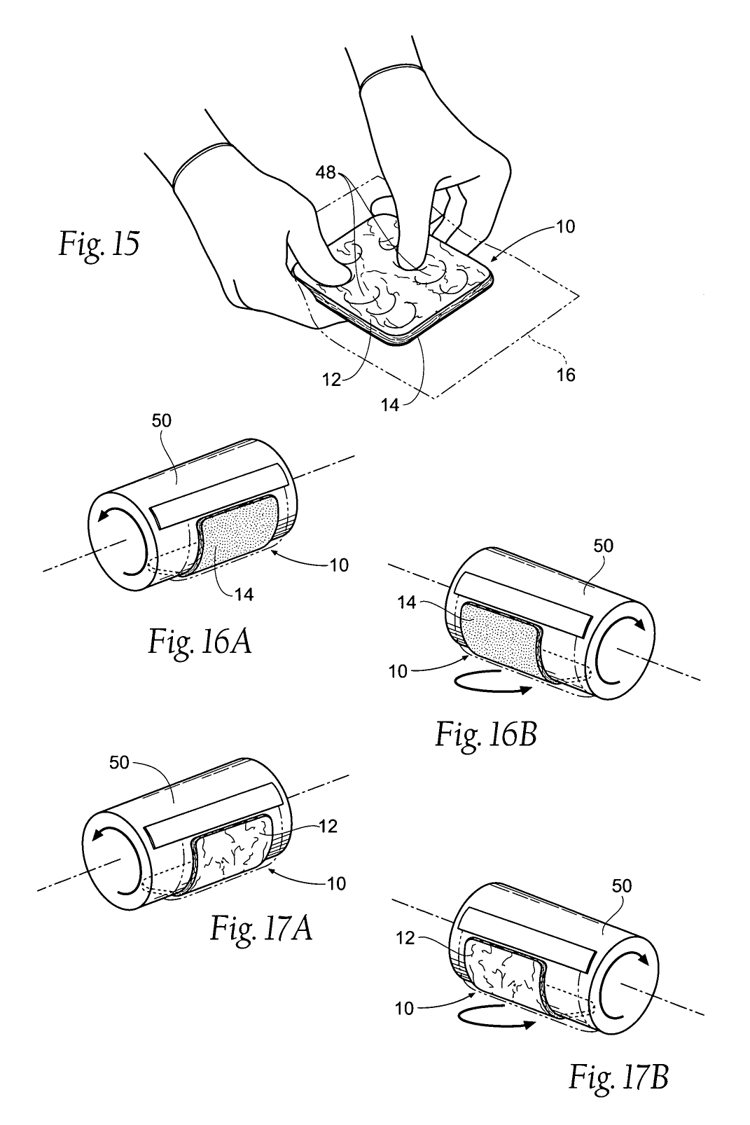

Desirably, as FIG. 15 shows, pre-conditioning can be performed with the pad assembly 10 sealed within its pouch 16. As FIG. 15 shows, maintaining the active face of the pad assembly 10 (i.e., the chitosan matrix 12) upright, manual repetitive digital impressions 48 of 1 to 1.5 mm depth can be applied over the entire surface. After application of the local pressure, and FIG. 16A shows, one edge of the square pad assembly 10, with active face remaining upright, can be attached to the side of a 7.5 cm diameter×12 cm long cylinder 50. The cylinder 50 is then rolled onto the pad assembly 10 to produce a 7.5 cm diameter concave in the pad assembly 10. The cylinder 50 can be released and the pad assembly 10 rotated 90° (see FIG. 16B) to enable another 7.5 cm diameter concave to be formed into the pad assembly 10. After this treatment, the pad assembly 10 can be flipped (i.e., with the backing 14 now upright) (see FIGS. 17A and 17B) to enable 90° offset, 7.5 cm diameter concaves to be formed in the backing 14 of the pad assembly 10. It is envisioned that the manipulation of the pad assembly 10 described here would be performed mechanically during its processing immediately prior to its loading and sealing into the final shipment package.

The mechanical pre-conditioning described above is not limited to the pre-conditioning by digital probing and/or drawing over cylinders. The pre-conditioning may also include any technique which provides for mechanical change inside any hydrophilic polymer sponge structure resulting in enhanced sponge flexural modulus without significant loss of sponge hemostatic efficacy. Such pre-conditioning would include mechanical manipulations of any hydrophilic sponge structure including, but not limited to, mechanical manipulations by bending, twisting, rotating, vibrating, probing, compressing, extending, shaking and kneading.

2. Controlled Macro-Texturing of a Hydrophilic Polymer Sponge Structure

Controlled macro-texturing (by the formation of deep relief patterns) in a given hydrophilic polymer sponge structure can achieve improved flexibility and compliance, without engendering gross failure of the pad assembly 10 at its time of use. With regard to the chitosan matrix 12, the deep relief patterns can be formed either on the active surface of the chitosan matrix 12, or on the backing 14, or both sides.

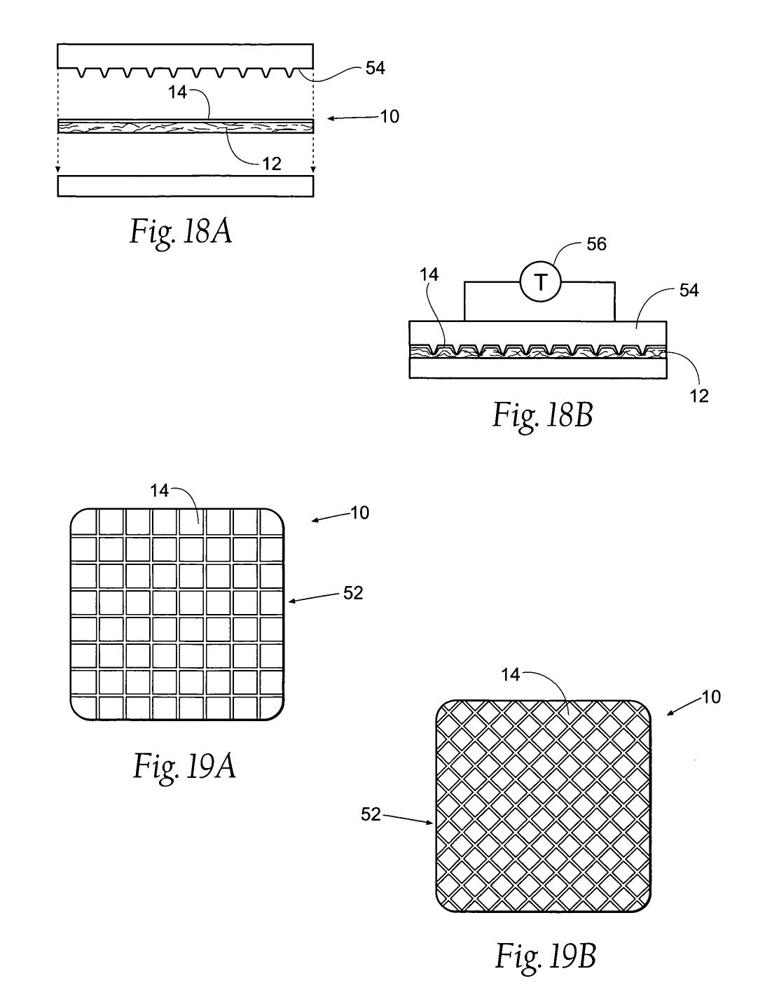

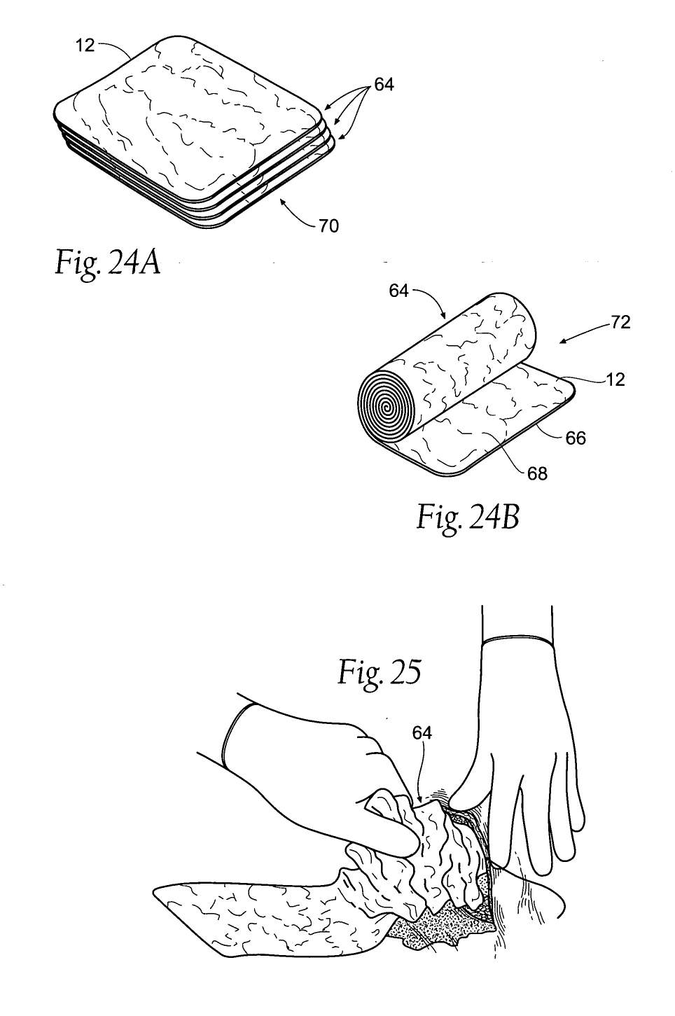

As FIGS. 18A and 18B show, deep (0.25-0.50 cm) relief surface patterns 52 (macro-textured surfaces) can be created in the pad assembly 10 by sponge thermal compression at 80° C. The sponge thermal compression can be performed using a positive relief press platen 54, which includes a controlled heater assembly 56. Various representative examples of the types of relief patterns 52 that can be used are shown in FIGS. 24A to 24D. The relief pattern negative is formed from a positive relief attached to the heated platen 54.

The purpose of the patterns 52 is to enhance dry pad assembly compliance by reduction in flexural resistance orthogonal to the relief 52, so that the relief pattern acts much like a local hinge to allow enhanced flexure along its length.

It is preferred that this relief 52 is applied in the backing 14 of the pad assembly 10 and not in the chitosan matrix 12, whose role is to provide hemostasis by injury sealing and promoting local clot formation. Macro-textured deep relief patterns 52 in the base chitosan matrix 12 can provide for loss of sealing by providing channels for blood to escape through the chitosan matrix 12.

In order to mitigate this possibility, alternative relief patterns 52 of the type shown in FIGS. 24E and 24F may be used in a base relief, which would be less likely to cause loss of sealing. It is therefore possible that the relief 52 may be use in the base of the matrix, however this is still less preferred compared to its use in the backing 14 or top surface of the matrix. By using two positive relief surfaces attached to top and bottom platens during sponge compression, it is also possible to apply relief patterns in top and bottom surfaces of the pad assembly 10 simultaneously. However it is more preferable that a single, deep relief is created by use of one positive relief in the top surface of the chitosan matrix 12.

### EXAMPLE 2

Mechanical flexure testing was carried out on a test pad assemblies (each 10 cm×10 cm×0.55 cm, with adherent backing 14—3M 1774T polyethylene foam medical tape 0.056 cm thick). One pad assembly 10 (Pad 1) comprised a chitosan matrix 12 having a predominantly vertical lamella structure (i.e., manufactured at a warmer relative freezing temperature, as described above). The other pad assembly 10 (Pad 2) comprised a chitosan matrix 12 having a predominantly horizontal, intermeshed lamella structure (i.e., manufactured at a colder relative freezing temperature, as described above).

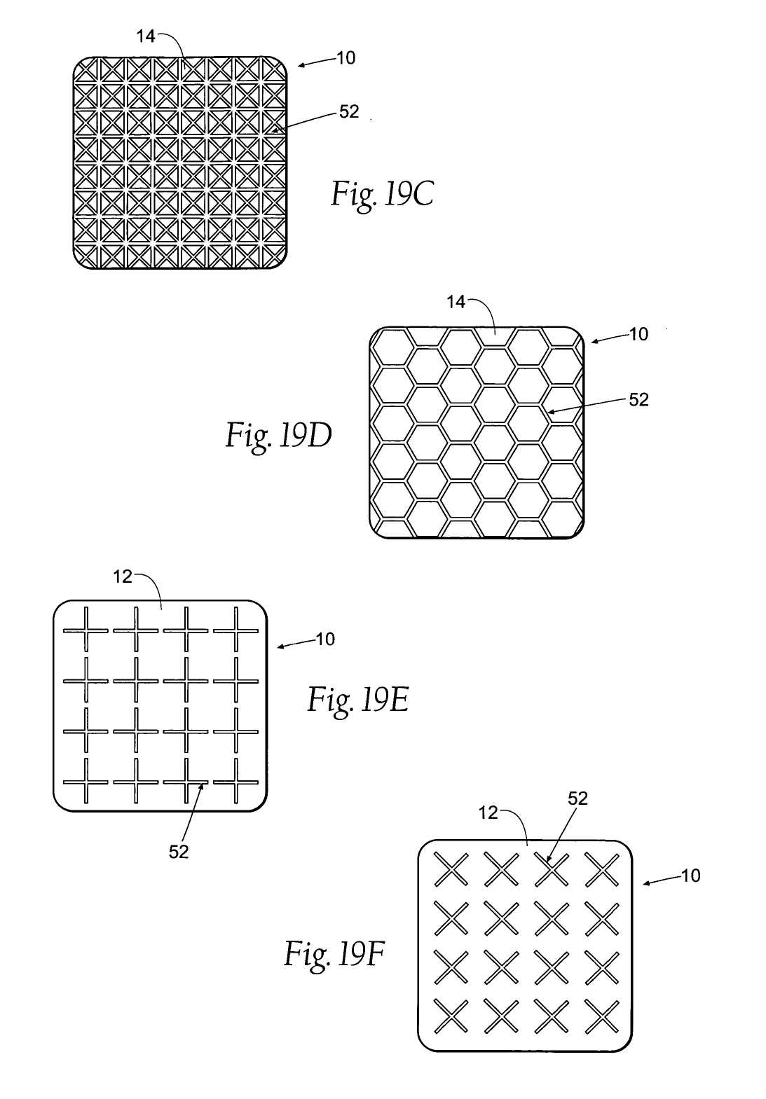

Each Pad 1 and 2 was cut in half. Two halves (5 cm×10 cm×0.55 cm) of each compressed chitosan pads 1 and 2, were locally compressed at 80° C. to produce the relief pattern on the backing 14, in the form of FIG. 19A. The other halves of the pads 1 and 2 were left untreated to be used as controls.

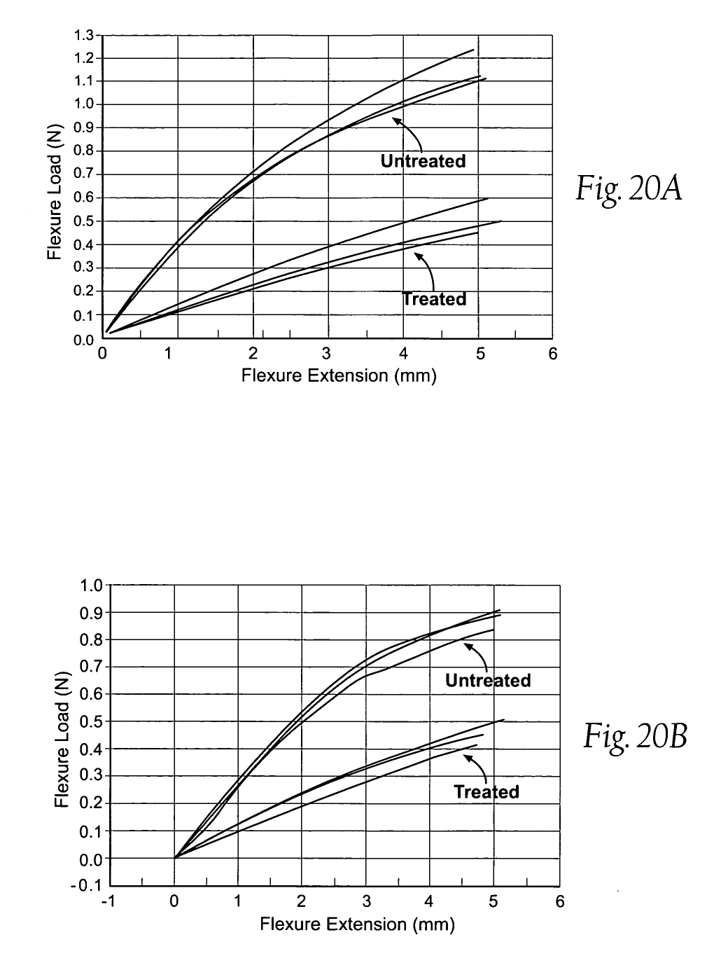

Three test pieces (10 cm×1.27 cm×0.55 cm) were cut from each half of the pad assembly 10 using a scalpel. These test pieces were subjected to three point flex testing. The test pieces had relief indentations 0.25 cm deep and 0.25 cm wide at the top surface. Each indentation was separated from its neighbor by 1.27 cm. Three point flex testing on an Instron uniaxial mechanical tester, model number 5844, with a 50 N load cell was performed to determine flexural modulus for the 0.55 cm thick test pieces with span 5.8 cm and crosshead speed of 0.235 cm/s. Flexural load was plotted against mid-point flexural displacement for the two pads 1 and 2 (treated and untreated) and are shown, respectively, in FIGS. 20A and 20B. Flexural moduli of treated versus untreated test pieces for Pads 1 and 2 (treated and untreated) are shown in Tables 9A and 9B, respectively.

The flexural testing demonstrates a significant improvement in flexibility with controlled macro-texturing of either type of the dry pad assembly 10.

TABLE 9A
Summary of Mechanical Testing of Pad Type 1 (Vertical Lamella)

| | Flexure load at | | Modulus (Young's - |
|---|---|---|---|
| | Maximum Flexure | Modulus (Automatic) | Cursor) |
| | stress (N) | (MPa) | (MPa) |
| 1 | 0.5 | 2.7 | 2.7 |
| 2 | 0.5 | 2.3 | 2.3 |
| 3 | 0.6 | 3.1 | 3.1 |
| 4 | 1.2 | 8.3 | 8.2 |
| 5 | 1.1 | 9.5 | 9.5 |
| 6 | 1.1 | 8.5 | 8.5 |

| | |
|---|---|
| Specimen Label 1 | Right Edge - Hinged w/Flex |
| Specimen Label 2 | Inside Right Edge - Hinged w/Flex |
| Specimen Label 3 | Middle - Hinged w/Flex |
| Specimen Label 4 | Middle - Control |
| Specimen Label 5 | Inside Left Edge - Control |
| Specimen Label 6 | Left Edge - Control |

TABLE 9B
Summary of Mechanical Testing of
Pad Type 2 (Horizontal Lamella)

| | Flexure load at | | Modulus (Young's - |
|---|---|---|---|
| | Maximum Flexure | Modulus (Automatic) | Cursor) |
| | stress (N) | (MPa) | (MPa) |
| 1 | 0.4 | 2.1 | 2.0 |
| 2 | 0.5 | 2.7 | 2.7 |
| 3 | 0.5 | 3.0 | 3.0 |
| 4 | 0.9 | 6.1 | 6.1 |
| 5 | 0.9 | 5.6 | 5.7 |
| 6 | 0.8 | 6.3 | 6.3 |

| | |
|---|---|
| Specimen Label 1 | Right Edge - Hinged |
| Specimen Label 2 | Inside Right Edge - Hinged |
| Specimen Label 3 | Middle - Hinged |
| Specimen Label 4 | Middle - Control |
| Specimen Label 5 | Inside Left Edge - Control |
| Specimen Label 6 | Left Edge - Control |

3. Controlled Formation of Vertical Channels in a Hydrophilic Polymer Sponge Structure

A controlled introduction of blood into, and through the bulk of a given hydrophilic polymer sponge structure, of which the chitosan matrix 12 is but one example, is desirable for improved initial structural compliance and also for longevity of resistance to structure dissolution. Controlled formation of vertical channels into a given hydrophilic polymer sponge structure can achieve improved flexibility and compliance, without engendering gross failure of the structure at its time of use.

A controlled introduction of blood into, and through the bulk of a hydrophilic polymer sponge structure is desirable for improved initial compliance of the structure and also for longevity of resistance to dissolution of the structure. Improved absorption of blood into a hydrophilic polymer sponge structure can be accomplished by the introduction of vertical channels into the structure. Channel cross sectional area, channel depth and channel number density can be controlled to ensure an appropriate rate of blood absorption and distribution of blood absorption into the hydrophilic polymer sponge structure. With respect to the chitosan matrix 12, typically, a 200% increase in chitosan matrix 12 mass associated with blood absorption from 5 g to 15 g can cause a flexural modulus reduction of near 72%, from 7 MPa to 2 MPa. Also, controlled introduction of blood into the chitosan matrix 12 can result in a more cohesive matrix.

This improvement in the strength of a hydrophilic polymer matrix is a consequence of reaction of blood components, such as platelets and erythrocytes, with the same matrix. After introduction of blood into the sponge structure and allowance for time for the sponge structure and blood components to react to produce a blood and hydrophilic polymer sponge structure “amalgam,” the subsequent sponge structure is resistant to dissolution in body fluids and cannot be dissolved readily, especially in the case of a chitosan acid salt matrix, by the introduction of saline solution. Typically, prior to the reaction between blood and the hydrophilic polymer sponge structure, especially in the case of a chitosan acid salt matrix, the introduction of saline causes rapid swelling, gelling and dissolution of the hydrophilic polymer sponge structure.

Still, excessive introduction of blood into a given hydrophilic polymer sponge structure such as the chitosan matrix 12 can result in fluidized collapse. Therefore, mean channel cross-sectional area, mean channel depth and channel number density should be controlled to ensure that rate of blood absorption does not overwhelm the structure of the hydrophilic polymer sponge structure.

Controlled distribution of vertical channels in the hydrophilic polymer sponge structure can be achieved during the freezing step of the sponge structure preparation, or alternatively it may be achieved mechanically by perforation of the sponge structure during the compression (densification) step.

During the base nucleated freezing step, vertical channels can be introduced in the freezing solution by super-saturation of the same solution with residual gas. The same gas nucleates bubbles at the base of the solution in the mold as it begins to freeze. The bubbles rise through the solution during the freezing step leaving vertical channels. Sublimation of the ice around the channels during the lyophilization preserves the channels within the resultant sponge matrix.

Alternatively, channels may also be formed during the freezing step by the positioning of vertical rod elements in the base of the molds. Preferably the molds are formed from, but are not limited to, a metallic element such as iron, nickel, silver, copper, aluminum, aluminum alloy, titanium, titanium alloy, vanadium, molybdenum, gold, rhodium, palladium, platinum and/or combinations thereof. The metallic rod elements are preferably formed from, but not limited to, a metallic element such as iron, nickel, silver, copper, aluminum, aluminum alloy, titanium, titanium alloy, vanadium, molybdenum, gold, palladium, rhodium or platinum and/or combinations thereof. The molds may also be coated with thin, inert metallic coatings such as titanium, chromium, tungsten, vanadium, nickel, molybdenum, gold and platinum in order to ensure there is no reaction with the acid component of the chitosan solution and the chitosan salt matrix. Thermally insulating coatings or elements may be used in conjunction with the metallic molds and vertical rod elements to control heat transfer in the molds and in the vertical rod elements. Although metallic molds and vertical metallic rod elements are preferable, plastic molds and vertical plastic mold rod elements can be a convenient alternative for creating channels. An advantage of the metallic molds and their metallic rod elements combined with local placement of thermally insulating elements is that they also provide opportunity for improved control of heat flow and structure within the freezing sponge structure. This improvement in heat flow control results from large thermal conductivity differences between thermally conducting and thermally insulating elements in the mold and also the ability to create local thermal gradients within the bulk of the hydrophilic polymer sponge structure solution through the rod elements.

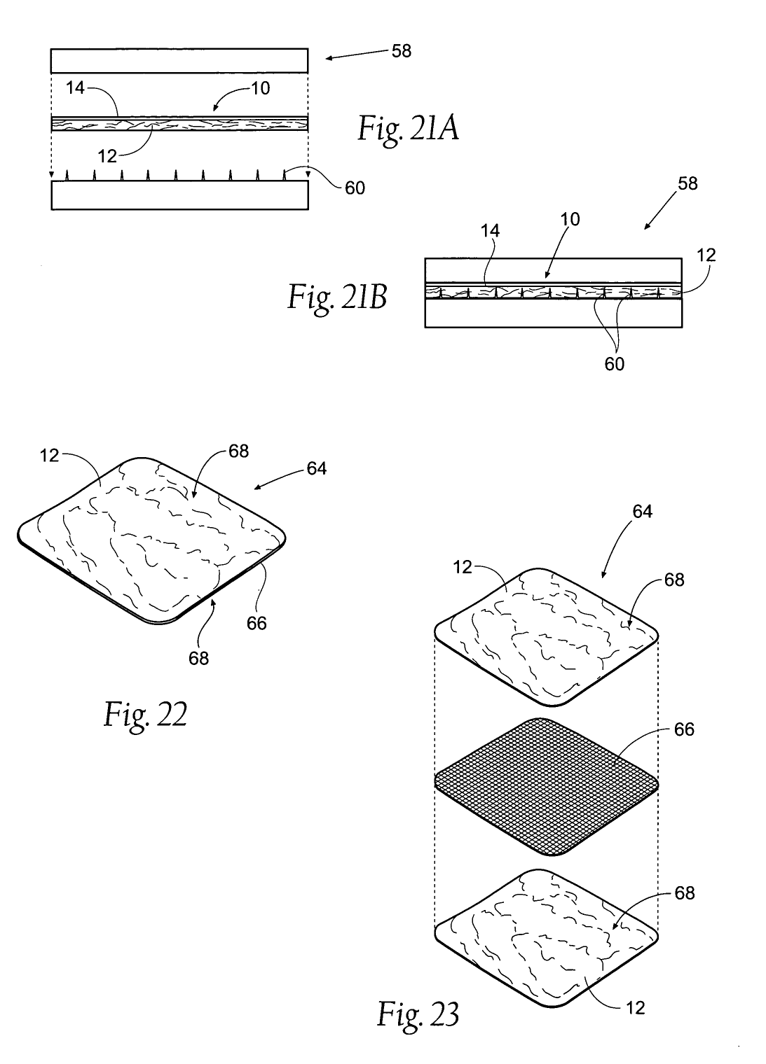

After lyophilization of the sponge structure, vertical channels can be introduced during the compression (densification) process. For example, as shown in FIGS. 21A and 21B, a compression fixture 58 carries a pincushion geometrical patterned device 60 for placing short (2.5 mm depth) equally spaced perforations 62 in the base of the sponge structure.

The intent of the perforations 62 is to allow local infiltration of blood at a slow controlled rate into and through the base of the hydrophilic polymer sponge structure. The purpose of this infiltration is first to allow for a more rapid flexural change in the matrix by plasticization of the dry sponge with blood. Secondly, it is intended to provide for a more uniform dispersion and mixing of blood through the matrix in order to stabilize the matrix to resist subsequent dissolution agents present within the body cavity. In the absence of the perforated base surface, it is seen after 1, 6, 16 and 31 minutes that blood only penetrates superficially into the sponge structure (<1.5 mm depth) while in the presence of the perforations that blood penetrates from 1.8 to 2.3 mm depth after 31 minutes. There is a resultant more rapid decrease in flexural modulus in the perforated matrix compared to a matrix without perforations.

II. Tissue Dressing Sheet Assembly

A. Overview

FIG. 22 shows a tissue dressing sheet assembly 64. Like the antimicrobial barrier pad assembly 10 previously described and shown in FIG. 1, the tissue dressing sheet assembly 64 is capable, in use, of adhering to tissue in the presence of blood or body fluids or moisture. The tissue dressing sheet assembly 64 can thus also be used to stanch, seal, and/or stabilize a site of tissue injury or trauma or access against bleeding or other forms of fluid loss. As for the antimicrobial barrier pad assembly 10, the tissue site treated by the tissue dressing sheet assembly 64 can comprise, e.g., arterial and/or venous bleeding, or laceration, or entrance/entry wound, or tissue puncture, or catheter access site, or burn, or suturing. The tissue dressing sheet assembly 64 can also form an anti-bacterial and/or anti-microbial and/or anti-viral protective barrier at or about the tissue treatment site.

FIG. 22 shows the tissue dressing sheet assembly 64 in its condition prior to use. As FIG. 23 best shows, the tissue dressing sheet assembly 64 comprises a sheet 66 of woven or non-woven mesh material enveloped between layers of a tissue dressing matrix 68. The tissue dressing matrix 68 impregnates the sheet 66. The tissue dressing matrix 68 desirably comprises a chitosan matrix 12 as described in connection with the antimicrobial barrier pad assembly 10. However, other hydrophilic polymer sponge structures can be used.

The size, shape, and configuration of the tissue dressing sheet assembly 64 can vary according to its intended use. The sheet assembly 64 can be rectilinear, elongated, square, round, oval, or composite or complex combinations thereof.

The tissue dressing sheet assembly 64 achieves rapid compliance of the hydrophilic polymer sponge structure in a bleeding field. The tissue dressing sheet assembly 64 is preferably thin (compared to the pad assembly 10), being in the range of between 0.5 mm to 1.5 mm in thickness. A preferred form of the thin reinforced structure of the sheet assembly 64 comprises a chitosan matrix 12 or sponge, at the typical chitosan matrix density of 0.10 to 0.20 g/cm3, reinforced by absorbable bandage webbing such as cotton gauze and the resultant bandage thickness is 1.5 mm or less.

The sheet assembly 64 can be prepared as a compact sheet form (e.g. 10 cm×10 cm×0.1 cm) for packaging in a multi-sheet flat form 70 (as FIG. 24A shows) or as an elongated sheet form (e.g. 10 cm×150 cm×0.1 cm) for packaging in a compact rolled sheet form 72 (as FIG. 24B shows). The sheet 66 provides reinforcement throughout the assembly 64, while also presenting significant specific hydrophilic polymer sponge structure surface area availability for blood absorption. The presence of the woven or non-woven sheet 66 also serves to reinforce the overall hydrophilic polymer sponge structure.

The sheet 66 can comprise woven and non-woven mesh materials, formed, e.g., from cellulose derived material such as gauze cotton mesh. Examples of preferred reinforcing materials include absorbent low-modulus meshes and/or porous films and/or porous sponges and/or weaves of synthetic and naturally occurring polymers. Synthetic biodegradable materials may include, but are not limited to, poly(glycolic acid), poly(lactic acid), poly(e-caprolactone), poly(β-hydroxybutyric acid), poly(β-hydroxyvaleric acid), polydioxanone, poly(ethylene oxide), poly(malic acid), poly(tartronic acid), polyphosphazene, polyhydroxybutyrate and the copolymers of the monomers used to synthesize the above-mentioned polymers. Naturally occurring polymers may include, but are not limited to, cellulose, chitin, algin, starch, dextran, collagen and albumen. Non-degradable synthetic reinforcing materials may include but are not limited to polyethylene, polyethylene copolymers, polypropylene, polypropylene copolymers, metallocene polymers, polyurethanes, polyvinylchloride polymers, polyesters and polyamides.

B. Use of the Tissue Dressing Sheet Assembly

The thin sheet assembly 64 possesses very good compliance and allows for excellent apposition of the hydrophilic polymer sponge structure (e.g., the chitosan matrix 12) immediately against the injury site. Also the reinforcement of the sheet enables the overall assembly to resist dissolution in a strong bleeding field. The sheet assembly 64 accommodates layering, compaction, and/or rolling—i.e., “stuffing” (as FIG. 25 shows)—of the hydrophilic polymer sponge structure (e.g., the chitosan matrix 12) within a wound site using pressure to further reinforce the overall structure against strong arterial and venous bleeding. By stuffing of the sheet structure over itself, as FIG. 32 shows, the interaction of the blood with the hydrophilic polymer (e.g., chitosan) infused within the webbing provides advantages for the application when the wounds are particularly deep or otherwise apparently inaccessible. The stuffing of the sheet assembly 64 into a bleeding wound and its compression on itself provide for a highly adhesive, insoluble and highly conforming bandage form.

C. Manufacture of the Tissue Dressing Sheet Assembly

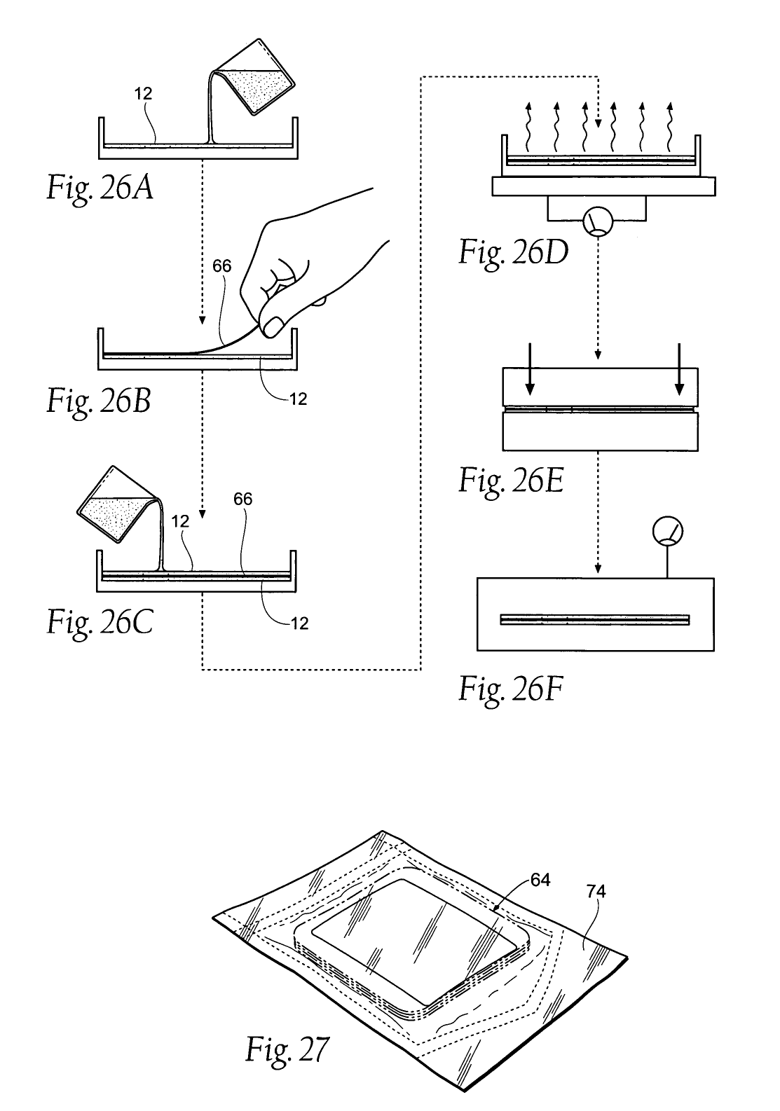

A tissue dressing sheet assembly 64 (10 cm×10 cm×0.15 cm), with chitosan matrix 12 density near 0.15 gm/cm3, can be prepared by filling 11 cm×11 cm×2 cm deep aluminum mold with a two percent (2%) chitosan acetate solution (see FIG. 26, Step A) to a depth of 0.38 cm.

As FIG. 26 (Step B) shows, the sheet 66—comprising, e.g., a layer of absorbent gauze webbing 10 cm×10 cm—can be placed over the top of the solution in the mold and allowed to soak with chitosan. The chitosan impregnates the sheet 66.

As FIG. 26 (Step C) shows, a further 0.38 cm depth of chitosan can be poured over the top of the impregnated gauze sheet 66.

As FIG. 26 (Step D) shows, the mold is placed in, e.g., a Virtis Genesis 25XL freeze dryer on a shelf at −30° C. The solution is allowed to freeze, after which the ice is sublimated by lyophilization.

As FIG. 26 (Step E) shows, the resultant gauze reinforced sheet assembly 64 is pressed between platens at 80° C. to a thickness of 0.155 cm. The pressed sheet assembly 64 is then baked at 80° C. for thirty minutes (FIG. 26, Step F). The resulting sheet assemblies can sterilized in a manner previously described. One or more sheet assemblies can be packaged within in a heat sealed foil lined pouch 74 or the like (see FIG. 27), either in sheet form or roll form for terminal sterilization and storage.

### EXAMPLE 3

### Flexural Characteristics of the Tissue Dressing Sheet Assembly

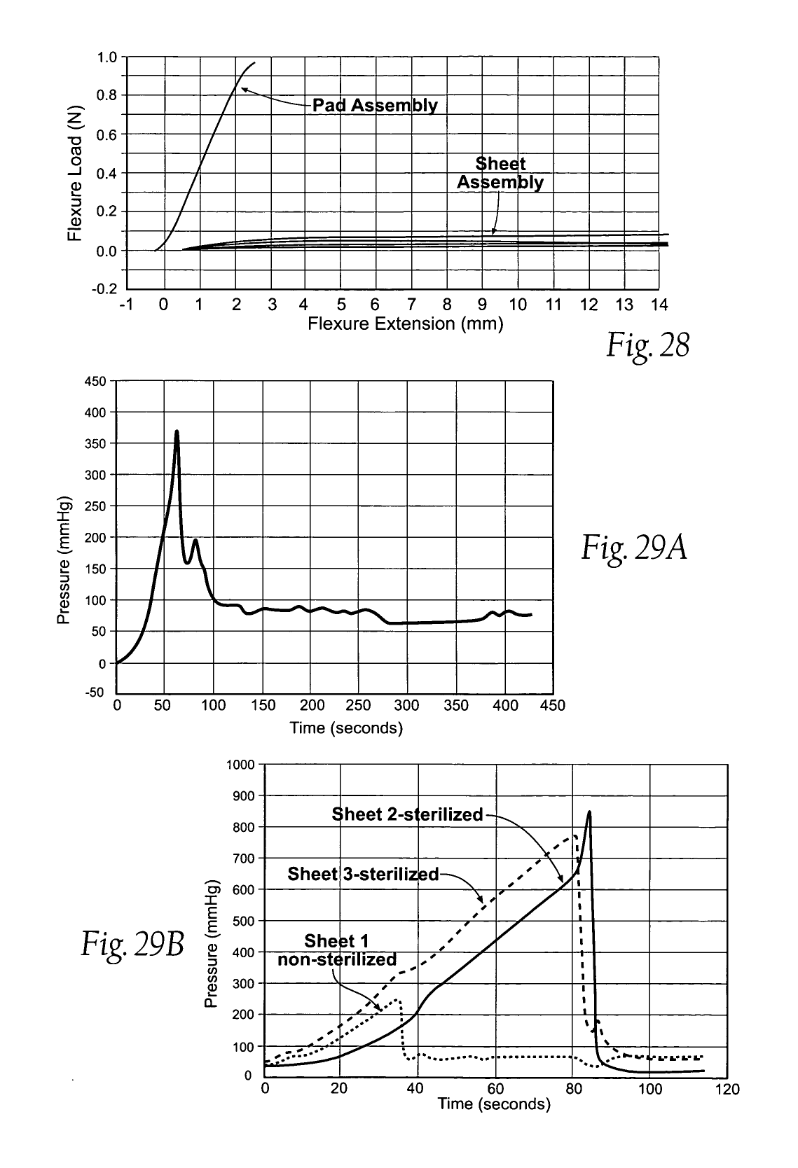

Flexural three point bend testing of a tissue dressing sheet assembly 64 was performed. The three point flexural testing was performed on an Instron uniaxial mechanical tester, model number 5844, with a 50 N load cell to determine flexural modulus test pieces with span 5.8 cm and crosshead speed of 0.235 cm/s. The results are shown in FIG. 28. FIG. 28 demonstrates that the 1.5 mm thick tissue dressing sheet assemblies that were tested are significantly more compliant than the 5.5 mm thick tissue dressing pad assemblies.

### EXAMPLE 4

### Adhesion Characteristics of the Tissue Dressing Sheet Assembly

Test pieces (5 cm×5 cm×0.15 cm) of the tissue dressing sheet assembly 64 were cut within ninety-six hours of their production. The sheet assembly 64 was not subjected gamma radiation sterilization before testing. The test pieces were soaked in citrated bovine whole blood for 10 seconds and immediately subjected to SAWS testing. During the test, three test pieces were layered together, presenting a composite chitosan density near 0.15 g/cm3. The result of this testing is shown in FIG. 29.

As FIG. 29A shows, the three layers of tissue dressing sheet assembly 64 held substantial physiological blood pressure of near 80 mmHg for an extended period (i.e., about 400 seconds). This indicates the presence of sealing and clotting.

Based upon experience with the pad assemblies, better adhesion/cohesion properties were expected to result after the tissue dressing sheet assembly 64 underwent gamma irradiation. FIG. 29B confirms this: after gamma-irradiation, three layers of tissue dressing sheet assembly 64 performed significantly like a 0.55 cm thick chitosan tissue pad 10.

III. Further Indications and Configurations for Hydrophilic Polymer Sponge Structures

The foregoing disclosure has focused upon the use of the antimicrobial barrier pad assembly 10 and the tissue dressing sheet assembly 64 principally in the setting of stanching blood and/or fluid loss at a wound site. Other indications have been mentioned and certain of these and other additional indications now will be described in greater detail.

Of course, it should be appreciated by now that the remarkable technical features that a compressed hydrophilic polymeric sponge structure, of which the chitosan matrix is but one example, possesses can be incorporated into dressing structures of diverse shapes, sizes, and configurations, to serve a diverse number of different indications. As will be shown, the shapes, sizes, and configurations that a given compressed hydrophilic polymer sponge structure (e.g., the chitosan matrix 12) can take are not limited to the pad assembly 10 and sheet assembly 64 described, and can transform according to the demands of a particular indication. Several representative examples follow, which are not intended to be all inclusive of limiting.

B. Antimicrobial Barriers

In certain indications, the focus of treatment becomes the prevention of ingress of bacteria and/or microbes through a tissue region that has been compromised, either by injury or by the need to establish an access portal to an interior tissue region. Examples of the latter situation include, e.g., the installation of an indwelling catheter to accommodate peritoneal dialysis, or the connection of an external urine or colostomy bag, or to accomplish parenteral nutrition, or to connect a sampling or monitoring device; or after the creation of an incision to access an interior region of the body during, e.g., a tracheotomy, or a laparoscopic or endoscopic procedure, or the introduction of a catheter instrument into a blood vessel.

In FIGS. 40 and 41, one representative embodiment of an antimicrobial gasket assembly 82 is shown. The gasket assembly 82 is sized and configured to be placed over an access site, and, in particular, an access site where an indwelling catheter 88 resides. The antimicrobial gasket assembly 82 includes a tissue adhering carrier component 84, to which an anti-microbial component is secured. Desirably, the anti-microbial component comprises the chitosan matrix 12 of the type previously described, which has undergone densification. Still, other types of a chitosan structure, or other hydrophilic polymer sponge structures, or tissue dressing matrixes in general can be used.

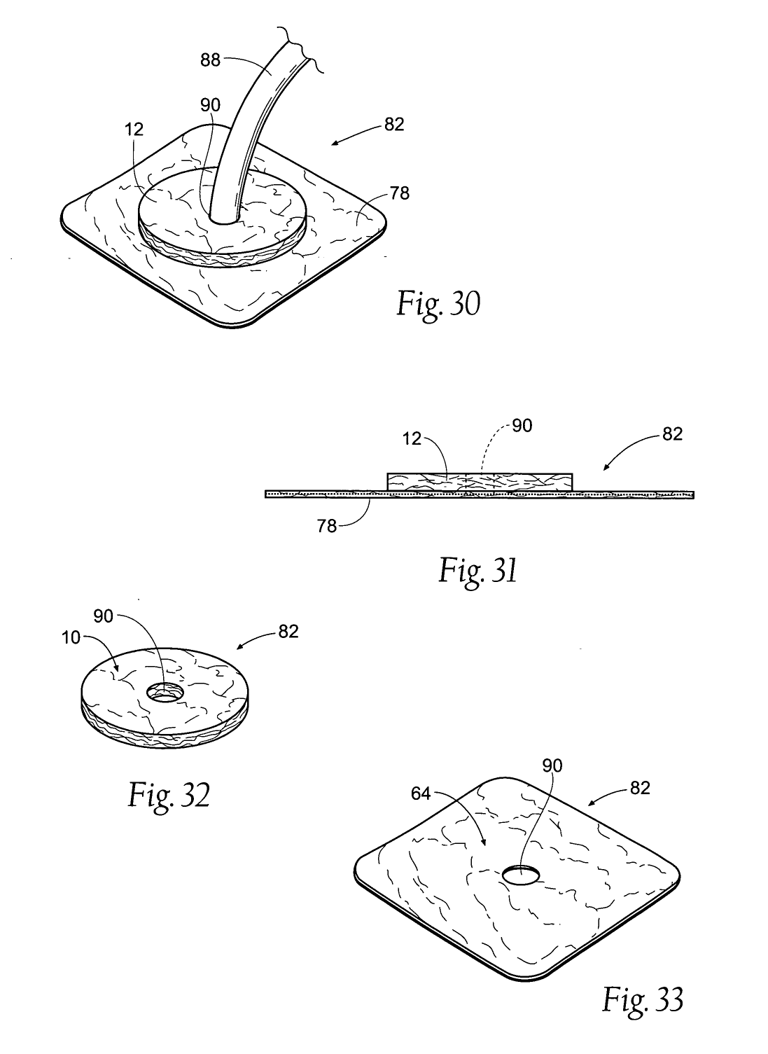

The carrier component 84 desirably includes an adhesive surface 86, to attach the anti-microbial component (desirably, the chitosan matrix 12) over the access site. In FIGS. 30 and 31, the anti-microbial component 12 and carrier 84 include a pass-through hole 90, which allows passage of the indwelling catheter 88 through it. In this arrangement, the interior diameter of the pass-through hole 90 approximates the exterior diameter of the indwelling catheter 88, to provide a tight, sealed fit. It should be appreciated that, in situations where there is only an incision or access site without a resident catheter, the anti-microbial component will not include the pass-through hole.

In an alternative arrangement (see FIG. 32), a antimicrobial barrier pad assembly 10 as previously described is sized and configured proportionate to the area of the access site to comprise an anti-microbial gasket assembly 82. In this configuration, the pad assembly 10 can be provided with a pass-through hole 90 to accommodate passage of an indwelling catheter, if present.

In another alternative arrangement (see FIG. 33), a tissue dressing sheet assembly 64 as previously described is sized and configured proportionate to the area of the access site to comprise an anti-microbial gasket assembly 82. In this configuration, the sheet assembly 64 can be provided with a pass-through hole 90 to accommodate passage of the indwelling catheter, if present.

### EXAMPLE 5

### Anti-Microbial Feature

The densified chitosan acetate matrix and diverse forms of dressings that can incorporate the densified chitosan acetate matrix have anti-microbial efficacy as demonstrated by in vitro testing, as summarized in Table 11.

TABLE 11
Results of USP 27<51> Testing of
the Densified Chitosan Acetate Matrix.

| | Log₁₀ Reduction at | | | | | | |
|---|---|---|---|---|---|---|---|
| | 0 | 24 | 48 | 72 | 7 | 14 | 28 |
| Organism | hrs | hrs | hrs | hrs | days | days | days |
| S. Aureus | 0.9 | 5.8 | 3.8 | 5.8 | 5.8 | 5.8 | 5.8 |
| P. Aeruginosa | 3.8 | 5.8 | 5.8 | 5.8 | 5.8 | 5.8 | 5.8 |
| E. coli | 0.0 | 2.8 | 5.1 | 5.1 | 5.1 | 5.1 | 5.1 |
| C. albicans | 5.5 | 5.5 | 5.5 | 5.5 | 5.5 | 5.5 | 5.5 |
| A. niger | 0.2 | −0.3 | 0.8 | 0.6 | −0.6 | −0.3 | −0.7 |

The excellent adhesive and mechanical properties of the densified chitosan matrix 12 make it eminently suitable for use in anti-microbial applications on the extremity (epidermal use) and inside the body. Such applications would include short to medium term (0-120 hour) control of infection and bleeding at catheter lead entry/exit points, at entry/exit points of biomedical devices for sampling and delivering application, and at severe injury sites when patient is in shock and unable to receive definitive surgical assistance.

### EXAMPLE 6

### In Vivo Testing of Topical Antimicrobial Efficacy

Further in vivo testing of the densified chitosan acetate matrix 12 was carried out and compared to similar dressings and treatments, specifically alginate dressing and Ag sulfadiazine. The testing was performed on male mice, strain BALB/c, approximately 6 weeks old and weighing approximately 20-25 grams. The lower portion of the mice were depilated and were anesthetized by injection of a 9:1 ratio of ketamine HCL to xylazine (100 mg/kg). Full thickness excisional wounds of desired size were cut down to, but not through, the panniculus carnosus.

The mice were infected with the Gram-negative species Pseudomonas aeruginosa [strain 19660] and Proteus mirabilis [strain 51393] that had been stably transduced with the entire bacterial lux operon to allow in vivo bioluminescence imaging. The strains were used for a bacterial culture, and 1 ml of the culture was used in 30-40 ml of sterile brain.heart infusion (BHI) media. The bacteria was grown to exponential growth phase for 2 hours in a 37° C. incubator with shaking. The O.D. of the bacterial suspension was measured against the BHI media and the desired suspension of bacteria was prepared accordingly.

Bioluminescence imaging was performed using a Hamamtsu CCD camera to detect the emitted light from wound infections of the mice.

The excisional wounds (5×5 mm) were inoculated with 50×10⁶ cells. In order to be able to measure luminescence transmission through the dressing pad assembly 10, a controlled thickness (1.6-2.4 mm) of densified chitosan matrix 12 structure was excised from the base surface of the dressing (nominally 5.5 mm thick) for use in the study. The chitosan matrix 12 test pieces used in the study were 10 mm×10 mm×2.1 mm in dimension. Three controls were used in the study: a positive control of silver sulfadiazine; a negative control of alginate sponge (10 mm×10 mm×2.0 mm); and another negative control of no treatment. All treatments were applied within 15 to 30 minutes of inoculation of the wound with bacteria.

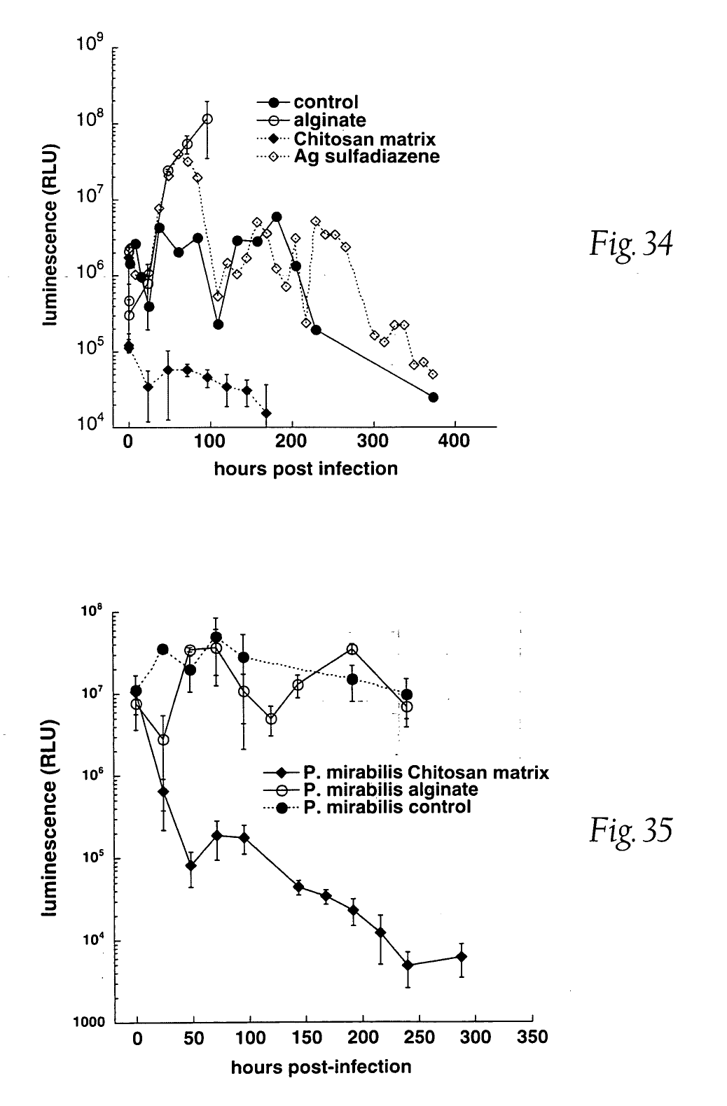

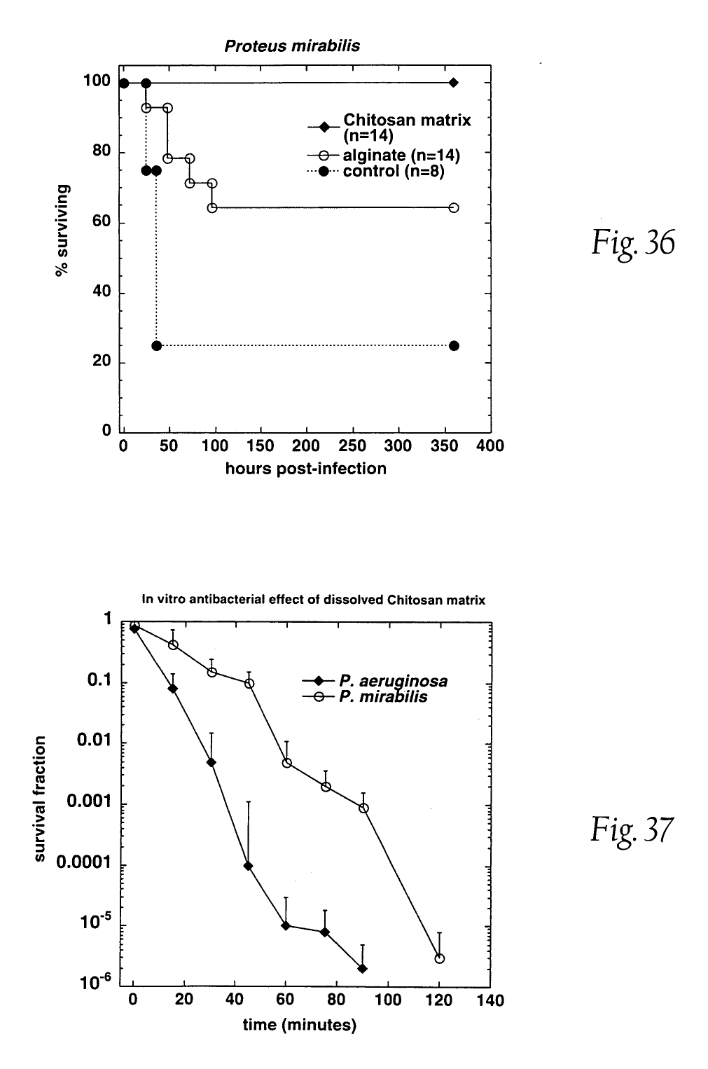

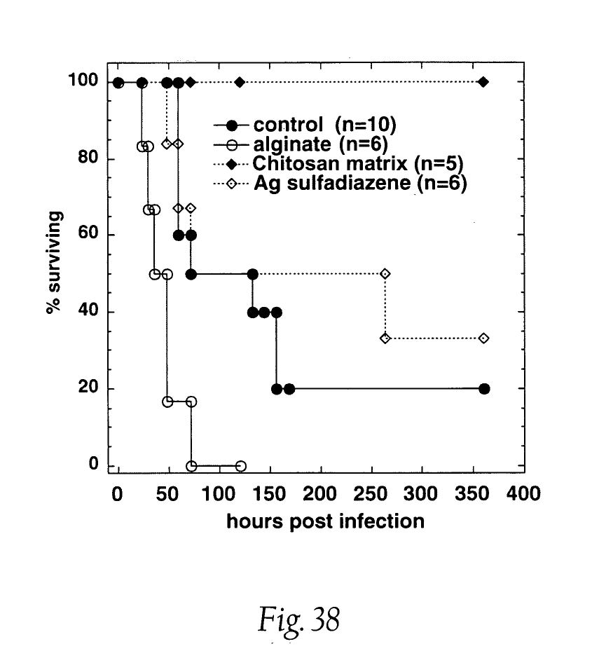

The densified chitosan matrix 12 sponge test pieces were first wetted with Na acetate buffer (pH 4) before application. They were adhesive and conformed very well to the injury. The alginate control was wetted with PBS solution prior to application. It too adhered well to the injury. The silver sulfadiazine cream (50 mg) was rubbed on the infected wound with a gloved finger. Animal survival was followed over 15 days with observations of bioluminescence emission and animal activity at regular intervals (8-16 hours). In the case of the densified chitosan matrix 12 group (N=5), all animals survived and showed significant survival advantage over alginate (P<0.01), over no treatment (P<0.005) and over silver sulfadiazine (P<0.005) (see FIG. 38). Also the densified chitosan matrix 12 was the only material to demonstrate significant loss in bioluminescence over the study period indicating marked bactericidal activity of this dressing (see FIGS. 34 and 35). None of the animals in the alginate group (N=6) survived beyond 5 days and the bioluminescent results indicated proliferation of the bacteria in this group (see FIGS. 35 and 36).

The data suggest that the densified chitosan matrix 12 rapidly kills bacteria in the wound before systemic invasion can take place, and is superior to alginate dressing and silver sulfadiazine that may both encourage bacterial growth in the short term. As shown in FIG. 37, the survival fraction of the bacteria when in contact with the densified chitosan matrix 12 diminishes quickly. Within 2 hours of treatment, nearly all of the bacteria had been destroyed by the chitosan matrix 12.

The chitsoan matrix 12 adheres well to wound areas and has rapid anti-microbial action. The combination of the anti-microbial and hemostatic qualities provides a superior wound dressing over the prior art, which is advantageous in early first aid treatment, such as in a combat, battlefield, or triage situation.

### IV. CONCLUSION

It has been demonstrated that a hydrophilic polymer sponge structure like the chitosan matrix 12 can be readily adapted for association with dressings or platforms of various sizes and configurations—in pad form, in sheet form, in composite form, in laminated form, in compliant form—such that a person of ordinary skill in the medical and/or surgical arts could adopt any hydrophilic polymer sponge structure like the chitosan matrix 12 to diverse indications on, in, or throughout the body.

Therefore, it should be apparent that above-described embodiments of this invention are merely descriptive of its principles and are not to be limited. The scope of this invention instead shall be determined from the scope of the following claims, including their equivalents.

We claim:

1. An antimicrobial barrier comprising:
a structure including a chitosan biomaterial.

2. The antimicrobial barrier of claim 1 wherein said structure further comprises a polymer sponge structure.

3. The antimicrobial barrier of claim 2 wherein said polymer sponge structure is a hydrophilic material.

4. The antimicrobial barrier of claim 3 wherein said polymer sponge structure further includes at least one of (i) micro-fracturing of a substantial portion of the structure by mechanical manipulation prior to use, or (ii) a surface relief pattern formed on a substantial portion of the structure prior to use, or (iii) a pattern of fluid inlet channels formed in a substantial portion of the structure prior to use.

5. An antimicrobial barrier according to claim 4, wherein the micro-fracturing results from at least one of bending, twisting, rotating, vibration, probing, compressing, extending, shaking, or kneading.

6. An antimicrobial barrier according to claim 4, wherein the surface relief pattern results from thermal compressing.

7. An antimicrobial barrier according to claim 4, wherein the structure includes a base surface and a top surface, and wherein the surface relief pattern is formed on the top surface and not on the base surface.

8. A tissue dressing according to claim 4, wherein the pattern of fluid inlet channels comprises perforations.

9. An antimicrobial barrier according to claim 1, wherein the structure includes a base surface and a top surface, and wherein a backing surface is located on the top surface.

10. A method of making an antimicrobial barrier as defined in claim 1.

11. A method of using an antimicrobial barrier as define in claim 1 to perform at least one of (i) stanch, seal, or stabilize a site of tissue injury, tissue trauma, or tissue access; or (ii) form an anti-microbial barrier; or (iii) form an antiviral patch; or (iv) intervene in a bleeding disorder; or (v) release a therapeutic agent; or (vi) treat a mucosal surface; or (vii) a combination thereof.

12. An antimicrobial barrier comprising:
a structure including a chitosan biomaterial, said structure having been densified by compression.

13. The antimicrobial barrier of claim 12 wherein said structure is compressed to a density of between 0.6 to 0.1 g/cm3.

14. A method of making an antimicrobial barrier as defined in claim 12.

15. A method of using an antimicrobial barrier as define in claim 12 to perform at least one of (i) stanch, seal, or stabilize a site of tissue injury, tissue trauma, or tissue access; or (ii) form an anti-microbial barrier; or (iii) form an antiviral patch; or (iv) intervene in a bleeding disorder; or (v) release a therapeutic agent; or (vi) treat a mucosal surface; or (vii) a combination thereof.

16. A method of reducing a bacterial count, the method comprising:
exposing a population of bacteria to a chitosan biomaterial.

17. A method of reducing a bacterial count to a non-invasive level, the method comprising:
exposing a population of bacteria to a chitosan biomaterial for a period of less than 2 hours.
